(19) United States
(12) Patent Application Publication
(10) Pub. No.: US 2008/0286287 A1
(43) Pub. Date: Nov. 20, 2008

(54) Methods and Agents for Modulating Cellular Activity

(75) Inventors: Vincenzo Carlo Russo, Parkville (AU); George Arthur Werther, Parkville (AU); Leon Ashley Bach, Melbourne (AU)

Correspondence Address: KNOBBE MARTENS OLSON & BEAR LLP, 2040 MAIN STREET, FOURTEENTH FLOOR, IRVINE, CA, 92614, US

(73) Assignee: Murdoch Childrens Research Institute, Parkville, Victoria (AU)

(21) Appl. No.: 11/910,406

(22) PCT Filed: Mar. 30, 2006
(86) PCT No.: PCT/AU2006/000428

§ 371 Date: Jul. 10, 2008

Related U.S. Application Data
(60) Provisional application No. 60/666,357, filed on Mar. 30, 2005.
(60) Provisional application No. 60/687,799, filed on Jun. 6, 2005.

(51) Int. Cl. A61K 39/395; C12N 5/06; A61K 38/02; A61P 35/00; A61K 31/7052

(52) U.S. Cl. 424/172.1; 435/375; 514/2; 514/44

(57) ABSTRACT

The present invention discloses the use of modified IGFBP-2 and agents that regulate the interaction between HBD of IGFBP-2 and EC or PC matrix components to prevent cellular proliferation invasion and/or migration (metastases).

### BACKGROUND OF THE INVENTION

1. Field of the Invention

The present invention relates generally to methods and agents, for modulating cellular proliferation, invasion and/or migration suitable for use in the treatment or prevention of cancer in animal including avian subjects. The present invention also provides drug targets and methods for screening for agents useful in the regulation of cellular activity and treatment or prevention of cancer. In one particular aspect, the present invention provides methods and agents for use in the treatment or prevention of cancers of the nervous system.

2. Description of the Prior Art

Bibliographic details of references in the subject specification are also listed at the end of the specification.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Advances in the production and synthesis of biopolymers such as nucleic acids, proteins and polysaccharides are facilitating the development of new therapeutic and diagnostic agents. Of particular interest are those agents which regulate cellular proliferation and behaviour. One important example of a condition which occurs in a subject with dysregulated cellular proliferation and behaviour is cancer.

The most widely applied treatment for primary and metastatic cancer (tumors) is a combination of surgery, radiotherapy and chemotherapy. The effectiveness of the treatment is limited particularly in the case of cancers of the brain, spine and peripheral nervous system because they must be minimally applied in order to minimise damage to sensitive brain or spinal chord tissue. Approximately 50% of brain tumours are benign but, like malignant tumors, residual tumor cells can proliferate again after treatment. Benign tumors of the brain can also cause death unlike tumors in other parts of the body, which are relatively harmless. Malignant nervous system tumors are currently the second leading cause of cancer deaths in young adults, children and in people over the age of sixty-five years. Unlike some other forms of cancer, such as skin or lung cancer there are no known behavioural risks for cancers of the nervous system so it is not possible to educate people to minimise their risk.

It is recognised that the above-described treatments are not ideal and there has been a concerted attempt to develop alternative treatments for cancer. Alternative treatments include the use of angiogenesis inhibiting agents to prevent the development of a blood supply to the growing tumour, immunotherapy, gene therapy and the targeted delivery of agents which prevent proliferation of dividing cells such as tumor cells. These agents are, like chemotherapeutic agents, ideally administered in a format which facilitates their access to the tumor in the spinal chord and/or brain and minimises unwanted effects.

The unwanted effects of treatment regimens include effects which can be predicted based on the mode of action of the drug. For example, chemotherapeutic agents will target all rapidly dividing cells in the body including cancer cells and cells of the bone marrow, hair follicle and gastrointestinal tract. A drug which has a very narrow window of activity, for example, one which targets cancer cells while leaving normal cells unharmed is a highly prized goal. One approach to this problem is the rational design or development of agents that target pathways which are critical for cellular proliferation, migration or invasion but are not essential for the proper working of beneficial physiological responses. Progress in this area largely depends upon insights provided by biomedical research directed towards understanding the interacting pathways involved in cellular responses.

The extracellular matrix (ECM) is a complex mixture of proteoglycans and glycoproteins which facilitate and modulate cell:cell and cell:molecule and cell:surface interations. Proteoglycans are large molecules comprising a core protein and glycosaminoglycan side chains (GAGs) covalently attached thereto. They are located at the cell surface, traversing the membrane, as part of the extracellular (EC)/pericellular (PC) matrix and secreted into the extracellular space. The ECM is therefore a subject of research to understand inter alia how it modulates cellular proliferation, invasion and migration.

The insulin-like growth factors (IGFs) were initially identified as modulators of growth hormone activity. They are now recognised as having various important roles in cellular development and homeostasis. For example, IGF-1 appears to influence neuronal structure and functions. It has been shown to have the ability to preserve nerve cell function and promote nerve growth in experimental studies. Because of these properties, recombinant human IGF-1 is in clinical trials for the treatment of amyotrophic lateral sclerosis.

Cellular responses to insulin-like growth factor (IGF) are modulated by a family of six insulin-like growth factor binding proteins (IGFBPs)(Firth et al., Endocr Rev 23.824-854, 2002). Modulation which has been shown to result in both up-regulation and down-regulation of receptor signalling is thought to be the result of IGF:IGFBP interactions that occur in the pericellular or extracellular space (Firth et al., 2002 (supra)). IGFBPs associate with components of the extracellular matrix (ECM) or cell surface via glycoproteins, collagens and integrins (Firth et al., 2002 (supra), Russo et al. Endocrinology 138:4858-4867, 1997). IGFBPs have been indicated in both inhibiting and potentiating the activity of IGF (Firth et al., 2002 (supra), Hoeflich et al. Endocrinology 140:5488-5496, 1999, Pereira et al. Cancer Res 64.977-984, 2004, Moore et al. Int J Cancer 105:1419-2003). IGF-independent activities of IGFBPs have also been described (Firth et al., 2002 (supra)). IGFBP-2 is the most abundant IGFBP in neuronal rich regions including the brain olfactory bulb. IGFBP-2 is also highly expressed in neoplasms of the nervous system (Firth et al., 2002 (supra), Wang et al. Cancer Res 63.4315-4321, 2003, Zhang et al. Brain Pathol 12:87-94, 2002, Sallinen et al Cancer Res 60:6617-6622, 2000, Nordqvist et al. Cancer Research 57:2611-2614, 1997, Elmlinger et al. Endocrinology 142:1652-1658, 2001, Wang et al. J Cutan Pathol 30:599-605, 2003). In the case of gliomas, which are the most common primary tumor of the central nervous system, over expression of IGFBP-2 leads to increased metalloproteinase expression and increased invasion of brain parenchyma (Wang et al., 2003 (supra)).

As indicated above, there is a need in the art for agents which regulate cellular proliferation and activity suitable for use in the treatment, prevention or diagnosis of conditions characterized by dysregulated cellular proliferation, such as cancer, or those characterized by having too few cells of the correct phenotype in a subject or tissue. Additionally, it would be desirable to have therapeutic agents which are capable of mediating their effects without significantly inhibiting beneficial physiological responses and particularly those responses which are beneficial in the relation to down-regulating tumor development.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of sequence identifiers is provided in Table 1. A sequence listing is provided after the claims.

In accordance with one aspect of the present invention, it has been determined that inhibition of IGFBP-2 regulated pathways down regulates cellular proliferation, invasion and/or migration. In one embodiment, suppression of the interaction between a heparin binding domain (HBD) of IGFBP-2 and components of the extracellular (EC) or pericellular (PC) matrix inhibits or at least reduces cellular proliferation, invasion and migration.

It is to be understood that unless otherwise indicated, the subject invention is not limited to specific formulations of components, manufacturing methods, dosage regimens, or the like, as such may vary. It is also to be understood that the terminology used herein is for the purpose of describing particular embodiments only and is not intended to be limiting. Specifically, the present invention is described with particular reference to the role of modulators of the interaction between the HBD of IGFBP-2 and components of the PC/EC matrix on or adjacent to neuroblastoma cells. However, HBDs are structural features of other members of the family of IGFBP proteins, such as IGFBP-3, IGFBP-5 and IGFBP-6 (Firth et al., 2002 (supra)) and the interaction of these molecules with components of the PC/EC matrix on or adjacent to any cell, not limited to neurocellular cancers or neuroblastoma cells is expressly contemplated. For the avoidance of doubt, in some embodiments, the invention encompasses the family of IGFBPs and variants thereof. In other embodiments modified IGFBP-2 agents are preferred. In other embodiments the treatment of neural cells is preferred.

As demonstrated herein, IGFBP-2 binds to proteoglycans and heparin-like domains in cellular matrix components and once bound to or in the vicinity of target cells at an appropriate level or concentration there promotes cellular proliferation, invasion and migration. Hence, in another embodiment of the invention, the delivery of an effective amount of IGFBP-2 or a functional variant, analog or mimetic thereof to cells of the nervous system is used to promote or enhance neural development or repair processes via up regulating cellular proliferation, invasion and/or migration. In some embodiments, the present agents modulate one or more IGFBP-2 dependent pathways in the absence of significant effects on IGF/IGF receptor dependent pathways. In other embodiments, the present agents modulate one or more IGFBP-2 dependent pathways and IGF/IGF receptor dependent pathways.

Accordingly, in one aspect the present invention provides a method for the treatment or prophylaxis of a cancer in an animal including avian subject, the method comprising administering an effective amount of an agent capable of down regulating the interaction between IGFBP-2 and components of the extracellular (EC) pericellular (PC) matrix. In some embodiments, the cancer is a cancer of the nervous system. In some embodiments, the interaction is specifically the interaction between HBD of IGFBP-2 and components of the EC/PC matrix. In other embodiments, the interaction is between the HBD of IGFBP-2 and an IGFBP-2 binding domain (IBD) of components of the EC/PC matrix. In some embodiments, the components of the EC/PC matrix are selected from the group comprising or consisting of heparin, aggrecan, proteoglycan, integrin laminin, fibronectin, vitronectin, and collagen such as collagen type IV. In other embodiments, glycoaminoglycan moieties of matrix components are targeted by the subject agents.

In some embodiments, the agent down regulates the activity of an HBD of IGFB-2. In other embodiments, the agent down regulates the activity of an IBD of one or more EC/PC matrix glycoprotein components. Down regulation may be direct or indirect. That is, agents may, for example, bind directly to an HBD or IBD and directly down regulate the interaction between these domains. Binding of the HBD to EC/PC components comprises electrostatic interaction mediated by anionic oligosaccharide moieties binding electrostatically to a positively charged HBD. By IBD is meant portions of EC/PC components which mediate binding to the HBD. As the skilled person will appreciate it is possible to determine the sequence of the IBD using strategies such as those developed to determine the heparin-antithrombin III interaction responsible for heparin's anticoagulant activity. See, for example, Iozzo Ann Rev Biochem 67:609-652, 1998 and Bernfield et al. Annu Rev Biochem 68:729-777, 1999.

Alternatively, agents may, for example, bind to regions of IGFBP-2 or components of the EC/PC matrix which effectively down regulate the HBD:IBD interaction. Other forms of indirect regulation are mediated by genetic agents such as a sense or antisense molecule, ribozyme, DNAzyme or ribonuclease-type complex.

Cancers of the nervous system include neuroepithelial tumors such as without limitation astrocytomas, gliomas, oligodenrogliomas, spongioblastomas, ependymomas, medulloblastomas, neuroblastomas, choroid plexus papillomas, gangliomas, gangliocytomas and pineal tumours; and non-neuroepithelial tumors such as without limitation meningiomas, pituitary tumors, craniopharyngiomas, neurilemmomas, schwannomas, acoustic neuroma, melanomas, CNS lymphomas, chordomas, Rathke Cleft Cyst, brain metastases and leptomeningeal carcinomatoses.

EC/PC and cell surface matrix components are selected from the group comprising inter alia aggrecan, laminin, fibronectin, vitronectin, and collagen type IV and proteoglycan, glycosaminoglycan or mucopolysaccharide structures including heparin.

The agents of the present invention may comprise polypeptide/peptide, carbohydrate/oligoaccharide/glycosaminoglycan or lipid components or a combination of these or their analogs, Synthetic or recombinant approaches to the production of polypeptides and glycopolypeptides are routinely available as shown for example in Sambrook, Molecular Cloning: A Laboratory Manual, 3^{rd} Edition, CSHLP, CSH, NY, 2001. Peptide or peptide derivatives with specific affinity for glycosaminoglycan molecules have been developed as described for example in U.S. Pat. No. 6,852,696 incorporated herein by reference. A number of synthetic methods are now available for the synthesis of glycosaminoglycans and heparin-like glycoaminoglycans and analogs thereof, including the automated solution or solid phase synthesis of libraries of defined glycosaminoglycan oligosaccharides as set out, for example, in U.S. Pat. No. 6,846,917. Alternatively, the agent comprises genetic components, such as a single or double stranded oligonucleotide, inhibitory nucleic acid molecules or aptamers. In a further embodiment, the agent may be an organic or inorganic compound. The agents may be derived from any source. They may, for example, be isolated from natural sources or produced synthetically, chemically/enzymatically or recombinantly.

In an illustrative embodiment, tumor cells are targeted and transfected with a vector or plasmid comprising a genetic sequence encoding all or part of IGFBP-2 wherein an HBD encoding region is modified such that the HBD domain no longer binds to EC/PC matrix components and wherein the vector or plasmid is capable of replacing all or part of the endogenous IGFBP-2 encoding region. Cells expressing such modified forms of IGFBP-2 or cells contacted with such modified forms of IGFBP are no longer stimulated by IGFBP-2 binding to EC/PC components to promote cellular proliferation, invasion and/or migration. In an exemplary embodiment, the basic residues of the HBD domain are substituted with non-basic residues. In other embodiments, any modification including deletion of the HBD domain, that generates a non-functional i.e. non-binding HBD is used.

Thus, in one embodiment, polynucleotides encoding IGFBP-2 form a basis for developing the agents of the present invention. The nucleotide sequence of human (h) IGFBP-2 is set forth in SEQ ID NO: 1. Variants of this sequence are contemplated and these are conveniently encompassed by reference to nucleotide sequences having at least 60% sequence identity thereto or to at least a region thereof comprising the HBD encoding sequence. Alternatively, variants are encompassed which hybridize under appropriate stringent conditions to all or a part comprising at least 10 contiguous nucleotides of the sequence set forth in SEQ ID NO:1 or its complement.

In some embodiments of the invention, antibodies are particularly useful agents for down regulating the interaction between the HBD of IGFBP-2 and EC/PC matrix components. Antibodies are generated using routine screening procedures, for example, such as those described in Harlow et al., Antibodies: A Laboratory Manual, Cold Spring Harbor Laboratory, 1988. For example, hybridoma cell lines expressing monoclonal antibodies are derived from cells resulting from the fusion of an immortal cell line with lymphocytes sensitised against a target region i.e. peptides comprising the HBD of the domain. Many variants on this general approach have now been developed and are encompassed. Antibodies may be modified to reduce the immunogenicity such as by humanisation or deimmunisation technologies. For example, once the critical binding sites of a murine antibody are identified they can be transposed to a corresponding human antibody structure. Phage antibodies may be obtained from libraries of phage antibodies having specificity for a plurality of antigens. Such libraries are constructed by known methods such as those described herein and in by Winter et al. Annual Review of Immunology 12:433-455, 1994 incorporated herein. The library is contacted with the antigen of hapten of interest and phage antibodies which bind to the hapten are isolated. The procedure is repeated until a population of phage antibodies having the desired specificity is obtained and the isolated phage antibodies are cloned by conventional methods by those of skill in the art. Another useful agent is an aptamer described briefly further herein.

In another embodiment, peptides comprising the HBD of IGFBP-2, or a variant thereof are used to inhibit the IGFBP-2 binding activity of ECM components. In another embodiment, the ECM binding activity of HBD of IGFBP-2 is inhibited using agents which specifically bind to the HBD of IGFBP-2. As the skilled artisan will appreciate, a wide range of agents useful in the methods of the present invention may be developed rationally or selected, without undue experimentation. For example, antibodies and their functional derivatives or analogues may be generated using routine procedures, peptides and oligonucleotides which selectively bind or which effectively inhibit the activity of the HBD of IGFBP-2 or the IGFBP-2 binding domain of ECM components are selected by what is now routine screening of collections of synthetic or naturally occurring molecules. Inhibitory agents may directly bind to the binding domains, or alternatively, allosteric agents may indirectly inhibit the interactions of the subject binding domains. Agents are also developed which are forms, fragments or variants including derivatives or analogs of IGFBP-2 including the HBD of IGFBP-2 or ECM components including the IGFBP-2 binding domain of ECM components. Thus, in some embodiments an agent comprising an ECM component or a variant thereof is used to down regulate IGFBP-2 regulated pathways. Specifically, for example, the agent comprises heparin or a variant or analog thereof which binds to the HBD of IGFBP-2 in the nervous system, prevents IGFBP-2 binding to ECM including pericellular or cell surface components and inhibits neurocellular proliferation, migration and invasion. In another embodiment, peptides comprising the HBD of IGFBP-2 bind to ECM including pericellular or cell surface components in the nervous system and inhibit IGFBP-2 redulated pathways. In one embodiment, the peptides inhibit the binding of endogenous IGFBP-2 to the cellular, including neurocellular surface.

Compositions comprising the agents of the present invention and one or more pharmaceutically acceptable carriers, diluents and/or excipients are specifically contemplated. Any such agents are further proposed for use in the manufacture of a medicament for the treatment or prevention of cancer. The subject agent are still further proposed for use in the manufacture of a medicament to modulate cellular proliferation, migration and invasion in subjects in need thereof.

In another embodiment, the present invention provides methods of screening for agents for use in the treatment or prophylaxis of cancer, comprising screening one or more agents for their ability to down regulate the interaction of the HBD of IGFBP-2 with EC/PC matrix —components. Any such agents will also be useful in vitro or in vivo to inhibit cellular proliferation, migration and invasion in the presence of ECM including pericellular or cell surface components. Traditional binding assays including solid phase assays an affinity chromatography may be used to test and develop suitable agents. In addition, biosensor and cytosensor analyse may also be employed.

TABLE 1
Summary of sequence identifiers

| SEQUENCE ID | |
|---|---|
| NO: | DESCRIPTION |
| 1 | Nucleotide sequence encoding human IGFBP-2 |
| | (from NCBI GenBank Accession No. NM-000597) |
| 2 | Amino acid sequence of human IGFBP-2 |
| | (from NCBI GenBank Accession No. NM-000597) |
| 3 | Mutagenic cassette for generating HBD mutant: |
| | forward primer |
| 4 | Mutagenic cassette for generating HBD mutant: |
| | reverse primer |
| 5 | Mutagenic cassette for generating HBD mutant: |
| | internal forward primer |
| 6 | Mutagenic cassette for generating HBD mutant: |
| | internal reverse primer |
| 7 | Mutagenic cassette for generating RGD mutant: |
| | forward primer |
| 8 | Mutagenic cassette for generating RGD mutant: |
| | reverse primer |
| 9 | Mutagenic cassette for generating RGD mutant: |
| | forward primer introducing a start codon and a |
| | XhoI restriction site |
| 10 | Mutagenic forward primer for generating mutations |
| | within a proteolytic cleavage domain |
| 11 | Mutagenic reverse primer for generating mutations |
| | within a proteolytic cleavage domain |

### BRIEF DESCRIPTION OF THE FIGURES

FIG. 1 is a photographic representation of an autoradiograph showing a decrease in the amount of radiolabelled IGFBP-2 binding to rat olfactory bulb membranes in the presence of increasing salt concentration.

FIG. 2 is a photographic representation of an autoradiograph showing binding of IGFBP-2 to cell membrane proteoglycans having a molecular mass in excess of 300 kDa and a decrease in binding thereto in the presence of chondroitin ABC lyase (Ch-ase) and Keratinase (K-ase). The radiolabelled band with a molecular mass in excess of 300 kDa is likely to be a radiolabelled IGF-1/IGFBP-2/proteoglycan complex which is competed out in the presence of unlabelled IGF-1 but not an analog of IGF-I with reduced affinity for IGFBPs (des(1-3)-IGF-1).

FIGS. 3 and 4 are graphical representations showing heparin binding domain (HBD) mediated binding of IGFBP-2 to aggrecan. Plates (96-well) were coated with 500 ng/well of aggrecan, saturated with 1% BSA, followed by incubation with native IGFBP (WT), HBD modified IGFBP-2 (the HBD modification is ₁₇₉PKKLRP₁₈₄ to ₁₇₉PNNLAP₁₈₄) (HBD) or RGE modified IGFBP-2 (the RGE modification is ₂₆₅RGD₂₆₇ to ₂₆₅RGE₂₆₇) (10 ng/well) for 2 hours at 37 C. Bound IGFBP-2 was detected by ¹²⁵I-IGF-I(1.5×10⁴ cpm). NS=non-specific binding was determined in the presence of an excess of unlabelled IGF-I (1 μg/ml). TB=total binding. In the absence of native or mutant IGFBP-2, ¹²⁵I-IGF-I binding to aggrecan was undetectable (data not shown). Binding of native and RGE-IGFBP-2 were decreased in the presence of increasing ionic strength (125-500 mM NaCl). The binding assay was as described in the Example 1. Each point was measured in triplicate in each of three experiments (***=p<0.001).

FIG. 5 is a graphical representation of data showing IGFBP-2 binding to ECM components. Native (WT) IGFBP-2 (10 ng/ml) binds to wells coated with 500 ng of aggrecan, heparin, laminin (LAM), fibronectin (FN), collagen type IV (COL-IV) and 300 ng of vitronectin (VN). The binding assay was as described in Example 1. Each point was measured in triplicate in each of three experiments.

FIG. 6A-E is a graphical representation of data demonstrating that IGFBP-2 binds to ECM components via its HBD. Native (WT), HBD and RGE-IGFBP-2 (10 ng/ml) bind to wells coated with 500 ng of aggrecan, heparin, laminin (LAM), fibronectin (FN), collagen type IV (COL-IV) and 300 ng of vitronectin (VN). The binding assay was as described in Example 1. Each point was measured in triplicate in each of three experiments (* p<0.05;***=p<0.001).

FIG. 7 is a graphical representation of data demonstrating that exogenous IGFBP-2 and modified mutants inhibit the mitogenic activity of IGF-I in SHEP cells. Thymidine incorporation assay was performed as described in Example 1. Each point was measured in triplicate in each of three experiments (**=p<0.01; ns=not significant).

FIG. 8 is a photographic representation and FIG. 9 is a graphical representation of data demonstrating that over-expression of IGFBP-2 and its modified mutants differentially modulate proliferation of SHEP cells. Conditioned medium from two clones for each of the SHEP cells over-expressing similar level of either WT, HBD or RGE-IGFBP-2 was analysed by comassie (top panel), immunoblotting with anti-IGFBP-2 (middle panel) and western ligand blotting (lower panel) as described in Example 1. Representative set is shown. FIG. 9) To assess the effects of over-expression of IGFBP-2 or its mutants on SHEP cell proliferation, SHEP cells clones expressing comparable amounts of IGFBP-2 or its mutants were isolated and grown in complete medium. Cell number was determined as described in Example 1. Each point was measured in triplicate in each of three experiments.

FIG. 10 is a graphical representation of data showing that overexpression of HBD-IGFBP-2 potently abolishes the IGF-1 response in SHEP cells. SHEP cells expressing IFGBP-2 (A), HBD-IGFBP-2 (B) or empty vector (C) were cultured in serum free medium (SF) in the presence or absence of IGF-1. In the absence of IGF-1 the cell number declined over time more in cells expressing HBD-IGFB-2 and cells expressing empty vector (C) than in cells expressing wild-type IGFBP-2 (A). IGF-1 elicited proliferation in SHEP cells containing IGFBP-1 and vector (C) but no response was detected in SHEP cells expressing HBD-IGFBP-2.

FIG. 11 is a graphical representation showing a significant difference in cell number increases over serum free control in SHEP cell clones expressing wild-type IGFBP-2 (WT), HBD-IGFBP-2 (HBD) and empty vector (CMV) over 24 hours. SHEP cells expressing HBD-IGFBP-2 show no measurable IGF-effect.

FIG. 12 is a graphical representation of results of MTT (Mosmann, J. Immunol. Methods, 16:65(1-2):55-63, 1983) proliferation assay showing that HBD-IGFB-2 is capable of inhibiting wild-type IGFBP-2 mediated proliferation. SHEP cells expressing WT-IGFBP-2 were grown in DMEM/10% FCS to reach 60% cell confluency, prior to media change with fresh DMEM/10% FCS (Time 0) and further culture for up to 72 hours (Time 72) in the presence or absence (black bars) of 1.5 μg/ml* of purified HBD-IGFBP-2 mutant. This mutant was added at Time 0 or 24 or 48 to allow exposure of WT-IGFBP-2 SHEP cells for the entire 72 hours (grey bars), or just for the last 48 (striped bars) or 24 hours (checked bars). In the latter treatments WT-IGFBP-2 SHEP cells were allowed to proliferate in optimal condition (DMEM/10% FCS) for the first 24 or 48 hours respectively.

FIGS. 13 and 14 are photographic and graphical representations, respectively, of data showing that over-expression of IGFBP-2 enhances invasion of neuroblastoma cells. Cell invasion was determined as described in Example 1. FIG. 13) microphotography of a representative set of filters is shown. FIG. 14) invasion index for each clone is shown. Each point was measured in duplicates in each of three experiments (*=p<0.05; **=p<0.01).

### BRIEF DESCRIPTION OF THE TABLES

Table 1 provides a description of the SEQ ID NOs provided herein.

Table 2 provides an amino acid sub-classification.

Table 3 provides exemplary amino acid substitutions.

Table 4 tabulates data showing the effect of mutagenesis on the affinity of IGFBP-2 for the IGFs. The HBD, ₁₇₉PKKLRP₁₈₄, was mutated to ₁₇₉PNNLAP₁₈₄ and the ₂₆₅RGD₂₆₇ domain was mutated to ₂₆₅RGE₂₆₇. Native and mutant IGFBP-2 were purified by IGF-I affinity chromatography and quantified by an IGFBP-2 ELISA or radioimmuno assay (RIA) as described in the Examples. IGF binding affinities of the modified IGFBP-2 mutants were similar to those of native IGFBP-2.

### DETAILED DESCRIPTION OF THE PREFERRED EMBODIMENTS

The present invention relates generally to methods and agents for regulating cellular activity such as proliferation, migration and invasion.

In accordance with the present invention it has now been determined that the heparin binding domain of IGFBP-2 mediates IGFBP-2 binding to components of the extracellular including pericellular or cell surface matrix and there promotes invasion, migration and/or proliferation of cells of the nervous system including neoplasms (tumors) of the nervous system. Thus, the heparin binding domain (HBD) of IGFBP-2 and the domains with which it interacts provide a new target for the development of agents which down-regulate IGFBP-2 regulated pathways useful in the prevention of cellular invasion, migration and/or proliferation in the nervous system and other tissues where IGFBP-2 is expressed. Specifically, as described herein pro-carcinogenic effects of IGFBP-2 are mediated after binding of IGBBP-2 to components of the EC/PC matrix and inhibited in the absence of a functional HBD of IGFBP-2. Conversely, up-regulation of IGFBP-2 is useful in enhancing cellular activity in the nervous system and other tissues where IGFBP-2 is expressed. The HBD is or is derived from the linker region of IGFBP-2 and is at amino acids 179-184 of IGFBP-2.

As used in the subject specification, the singular forms “a”, “an” and “the” include plural aspects unless the context clearly dictates otherwise. Thus, for example, reference to a “solvent” includes a single solvent, as well as two or more solvents; reference to “an agent” includes a single agent, as well as two or more agents; and so forth.

In describing and claiming the present invention, the following terminology is used in accordance with the definitions set forth below.

The terms “agent” or “medicament” refer to a chemical compound that induces a desired pharmacological and/or physiological effect. The term also encompass pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to peptides, polypeptides and proteins, carbohydrate and lipid molecules and combinations thereof such as glycopeptides and glycolipids as well as genetic molecules such as RNA, DNA and mimetics and chemical analogs thereof.

The term “modified” in the phrase “modified IGFBP” is meant to distinguish the subject molecules from variants which are essentially functional versions of the subject molecules as described further herein. The “modified” versions have lost one or more functions compared to a reference molecule such as a wild-type IGFBP molecule. For example, HBD-IGFBP-2 is a modified IGFBP-2 that has a non-functional HBD. In some embodiments, IGFBP-2 or HBD-IGFBP-2 is modified by the inactivation of proteinase cleavage sites. In come embodiments, IGFBP-2 is modified by alteration, mutation, deletion or all or part of the N-terminal and/or C-terminal regions in order to generate a molecule which binds to the IBD domain of EC or PC matrix but which fails to bind IGF. Modified molecules are generated using the same techniques used to generate variant molecules. Variants of modified IGFBP-2 molecules are, of course, expressly contemplated.

The term “modulator” is an example of an agent which regulates interaction between IGFBP-2 and EC/PC matrix components. The term “up-regulates” encompasses enhancing the interaction or the consequences of the interaction, while “down-regulates” encompasses reducing the level or frequency of interaction or reducing or ablating the binding affinity of the interacting portions. “Agonists” directly or indirectly up-regulate the interaction while “antagonists” directly or indirectly down regulate the interaction. The present invention contemplates, therefore, agents useful in modulating IGFBP-2 dependent pathways.

By “effective amount,” in the context of modulating an activity or of treating or preventing a condition is meant the administration of that amount of active to an individual in need of such modulation, treatment or prophylaxis, either in a single dose or as part of a series, that is effective for modulation of that effect or for treatment or prophylaxis of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, emide, prodrug or derivative of a compound as provided herein is a salt, ester, amide, prodrug or derivative that this not biologically or otherwise undesirable.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease. Generally, such a condition or disorder is a cancer. Thus, for example, the present method of “treating” a patient with cancer or with a propensity for one to develop encompasses both prevention of the condition, disease or disorder as well as treating the condition, disease or disorder. In any event, the present invention contemplates the treatment or prophylaxis of any cancer-like condition including without limitation cancers of the nervous system include neuroepithelial tumors such as astrocytomas, gliomas, oligodendroglioma, spongioblastomas, ependymomas, medulloblastomas, neuroblastomas, choroid plexus papillomas, gangliomas, gangliocytomas and pineal tumours; and non-neuroepithelial tumors such as without limitation meningiomas, pituitary tumors, craniopharyngiomas, neurilemmomas, schwannomas, acoustic neuroma, melanomas, CNS lymphomas, chordomas, Rathke Cleft Cyst, brain metastases and leptomeningeal carcinomatoses.

“Patient” and “subject” as used herein refers to an animal, preferably a mammal and more preferably human who can benefit from the pharmaceutical formulations and methods of the present invention. There is no limitation on the type of animal that could benefit from the presently described pharmaceutical formulations and methods. A patient regardless of whether a human or non-human animal may be referred to as an individual, subject, animal, host or recipient. The compounds and methods of the present invention have applications in human medicine, veterinary medicine as well as in general, domestic or wild animal husbandry. For convenience, an “animal” includes an avian species such as a poultry bird, an aviary bird or game bird. The preferred animals are humans or other primates, livestock animals, laboratory test animals, companion animals or captive wild animals. Examples of laboratory test animals include mice, rats, rabbits, guinea pigs and hamsters. Rabbits and rodent animals, such as rats and mice, provide a convenient test system or animal model. Livestock animals include sheep, cows, pigs, goats, horses and donkeys. Non-mammalian animals such as avian species, zebrafish and amphibians are also contemplated,

The agents of the present invention may be large or small molecules, nucleic acid molecules (including antisense or sense molecules), or hybrid molecules such as RNAi- or siRNA-complexes, ribozymes or DNAzymes or peptides, polypeptides or proteins, glycoproteins, glycolipids, lipids or oligosaccharide molecules.

The present invention contemplates, therefore, methods of screening for agents which modulate the interaction between IGFBP-2 and one or more components of the EC/PC matrix. The methods comprise, for example, contacting a candidate agent with one or more components of the interaction between IGFBP-2 and EC/PC components. The interacting molecules are referred to herein as a “target” or “target molecule”. The screening procedure includes assaying (i) for the presence of a complex between the agent and the target, or (ii) an alteration in the expression levels of nucleic acid molecules encoding the target if in nucleic acid form. One form of assay involves competitive binding assays. In such competitive binding assays, the target is typically labeled. Free target is separated from any putative complex and the amount of free (i.e. uncomplexed) label is a measure of the binding of the agent being tested to target molecule. One may also measure the amount of bound, rather than free, target. It is also possible to label the agent rather than the target and to measure the amount of agent binding to target in the presence and in the absence of the drug being tested. Such compounds may inhibit the target interaction which is useful, for example, in finding inhibitors of cancer progression.

Accordingly, the present invention provides a method of screening for agents useful in the treatment of cancer comprising contacting a putative agent with the HBD domain of IGFBP-2 to determine the presence of a complex between the agent and the target.

“Analogs” contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecule or their analogs. In relation to oligosaccharides or glycoconjucates the term analog encompasses modification to the number and composition of glycoaminoglycan chains, the number and composition is disaccharide repeat units, and the degree of sulphation or number of uronic acid residues or their derivatives.

By “biologically active portion” is meant in relation to polypeptide molecules, a portion of a full-length peptide or polypeptide which portion retains an activity of the full length molecule. As used herein, the term “biologically active portion” includes deletion mutants and peptides, for example, of at least about 8, 9, 10, 11, 12, 13, 14, 15, 16, 17, 18, 19, 20, 21, 22, 23, 24, 25, 30, 40, 50, 60, 70, 80, 90, 100, 120, 150, 300, 400, 500, 600, 700, 800, 900, 1000 contiguous amino acids, which comprise an activity of a parent molecule. Portions of this type may be obtained through the application of standard recombinant nucleic acid techniques or synthesized using conventional liquid or solid phase synthesis techniques. For example, reference may be made to solution synthesis or solid phase synthesis as described, for example, in Atherton et al., Peptide Synthesis, Chapter 9, “Synthetic Vaccines” edited by Nicholson and published by Blackwell Scientific Publications. Alternatively, peptides can be produced by digestion of proteins or glycoproteins with proteinases such as endoLys-C, endoArg-C, endoGlu-C and staphylococcus V8-protease. The digested fragments can be purified by, for example, high performance liquid chromatographic (HPLC) techniques. Recombinant nucleic acid techniques can also be used to produce such portions. In relation to proteoglycans and other components of EC/PC matrix, a biologically active fragment means portions of the retain IGFBP-2 and specifically HBD binding capability.

By “derivative” is meant a polypeptide that has been derived from the basic sequence by modification, for example by enzymatic cleavage, conjugation or complexing with other chemical moieties or by post-translational modification techniques as would be understood in the art. The term “derivative” also includes within its scope variants i.e., alterations that have been made to a parent sequence including additions, or deletions that provide for functionally equivalent molecules. Accordingly, in some embodiments, the term derivative encompasses molecules that will take part in the presently described interaction between IGFBP-2 and PC/EC matrix components and specifically HBD:IBD interactions. In an exemplary embodiment peptides comprising the amino acid sequence of the HBD i.e. at least about amino acids 179 to 184 of IGFBP-2 are conjugated to a delivery vehicle which provides administration to the target cells such as a neural cell.

When in nucleic acid form, a functional derivative comprises a sequence of nucleotides having at least 60% similarity to the target molecule or portions thereof. A “part” or “portion” of a nucleic acid molecule is defined as having a minimal size of at least about 10 nucleotides or preferably about 13 nucleotides or more preferably at least about 20 nucleotides and may have a minimal size of at least about 35 nucleotides. This definition includes all sizes in the range of 10-35 nucleotides including 10, 11, 12, 13, 14, 15, 16, 17, 18, 19, 20, 21, 22, 23, 24, 25, 26, 27, 28, 29, 30, 31, 32, 33, 34 or 35 nucleotides as well as greater than 35 nucleotides including 50, 100, 300, 500, 600 nucleotides or nucleic acid molecules having any number of nucleotides within these values.

In the context of this invention, “hybridization” means the pairing of complementary strands of oligomeric compounds. For example, adenine and thymine are complementary nucleobases which pair through the formation of hydrogen bonds. Hybridization can occur under varying circumstances as known to those of skill in the art.

The phrase “hybridizing specifically to” and the like refer to the binding or hybridization of a molecule only to a particular nucleotide sequence under stringent conditions when that sequence is present in a complex mixture (e.g., total cellular) DNA or RNA.

By “matrix metalloproteinase” or “MMP” is meant members of a large family of zinc-dependent proteases expressed by cells in response to growth factors or noxious stimuli and which are involved, in their active form, in tissue remodeling by breaking down matrix proteins such as collagens, fibrinogens proteoglycans, elastins, decorins and actins. The levels or activity of MMPs can be detected quantitatively or qualitatively by polymerase chain reaction (PCR) with primers specific for specific MMP mRNAs, by immunohistochemistry, ELISA or assays which detect MMP proteolytic activity.

The terms “genetic material”, “genetic forms”, “nucleic acids”, “nucleotide” and “polynucleotide” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule. RNA forms of the genetic molecules of the present invention are generally mRNA or iRNA including siRNAs. The genetic form may be in isolated form or integrated with other genetic molecules such as vector molecules and particularly expression vector molecules. The terms “nucleotide sequence”, “polynucleotide” and “nucleic acid molecule” may be used herein interchangeably and encompass polynucleotides in which one or more nucleotides have been added or deleted, or replaced with different nucleotides. In this regard, it is well understood in the art that certain alterations inclusive of mutations, additions, deletions and substitutions can be made to a reference nucleotide sequence whereby the altered polynucleotide retains the biological function or activity of the reference polynucleotide. The term “variant” also includes naturally-occurring allelic variants. The term “variant” refers to nucleotide sequences displaying substantial sequence identity with a reference nucleotide sequences or polynucleotides that hybridize with a reference sequence under stringency conditions that are defined hereinafter.

“Percentage similarity” between a particular sequence and a reference sequence (nucleotide or amino acid) is at least about 60% or at least about 70% or at least about 80% or at least about 90% or at least about 95% or above such as at least about 96%, 97%, 98%, 99% or greater. Percentage similarities or identities between 60% and 100% are also contemplated such as 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

The terms “polypeptide” or “proteinaceous molecule” refer to a polymer of amino acids and its equivalent and does not refer to a specific length of the product, thus, peptides, oligopeptides and proteins are included within the definition of a polypeptide. This term also does not exclude modifications of the polypeptide, for example, glycosylations, prenylatrion, acetylations, phosphorylations and the like. Soluble forms of the subject proteinaceous molecules IGFBP-2, are particularly useful. Included within the definition are, for example, polypeptides containing one or more analogs of an amino acid including, for example, unnatural amino acids or polypeptides with substituted linkages. The term “polypeptide variant” refers to polypeptides which are distinguished from a reference polypeptide by the addition, deletion or substitution of at least one amino acid residue. In certain embodiments, one or more amino acid residues of a reference polypeptide are replaced by different amino acids. It is well understood in the art that some amino acids may be changed to others with broadly similar properties without changing the nature of the activity of the polypeptide (conservative substitutions).

By “obtained from” means derived from, either directly or indirectly.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, H is, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, Calif., USA) using standard defaults as used in the reference manual accompanying the software.

“Stringency” as used herein refers to the temperature and ionic strength conditions, and presence or absence of certain organic solvents, during hybridization. The higher the stringency, the higher will be the observed degree of complementarity between sequences. “Stringent conditions” as used herein refers to temperature and ionic conditions under which only polynucleotides having a high proportion of complementary bases, preferably having exact complementarity, will hybridize. The stringency required is nucleotide sequence dependent and depends upon the various components present during hybridization, and is greatly changed when nucleotide analogues are used. Generally, stringent conditions are selected to be about 10 to 20° C. less than the thermal melting point (Tₘ) for the specific sequence at a defined ionic strength and pH. The Tₘ is the temperature (under defined ionic strength and pH) at which 50% of a target sequence hybridizes to a complementary probe.

It will be understood that an oligonucleotide will hybridize to another oligonucleotide under at least low stringency conditions, preferably under at least medium stringency conditions and more preferably under high stringency conditions. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. Medium stringency conditions include and encompass from at least about 16% v/v to at least about 30% v/v formamide and from at least about 0.5 M to at least about 0.9 M salt for hybridization at 42° C., and at least about 0.5 M to at least about 0.9 M salt for washing at 42° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 42° C. High stringency conditions include and encompass from at least about 31% v/v to at least about 50% v/v formamide and from at least about 0.01 M to at least about 0.15 M salt for hybridization at 42° C., and at least about 0.01 M to at least about 0.15 M salt for washing at 42° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. Other stringent conditions are well known in the art. A skilled addressee will recognize that various factors can be manipulated to optimize the specificity of the hybridization. Optimization of the stringency of the final washes can serve to ensure a high degree of hybridization. For detailed examples, see CURRENT PROTOCOLS IN MOLECULAR BIOLOGY (supra) at pages 2.10.1 to 2.10.16 and MOLECULAR CLONING. A LABORATORY MANUAL (Sambrook, et al., eds.) (Cold Spring Harbor Press 1989) at sections 1.101 to 1.104.

By “substantially complementary” it is meant that an oligonucleotide or a subsequence thereof is sufficiently complementary to hybridize with a target sequence. Accordingly, the nucleotide sequence of the oligonucleotide need not reflect the exact complementary sequence of the target sequence. In a preferred embodiment, the oligonucleotide contains no mismatches and with the target sequence.

Chemical, polypeptide or genetic agents may be identified through a wide range of screening protocols familiar to those of skill in the art.

High-throughput screening protocols are well used such as those described in Geysen (International Publication No. WO 84/03564). Briefly, large numbers of small peptide test compounds are synthesized on a solid substrate, such as plastic pins or some other surface. Bound polypeptide is detected by various methods. A similar method involving peptide synthesis on beads, which forms a peptide library in which each bead is an individual library member, is described in U.S. Pat. No. 4,631,211 and a related method is described in International Publication No. WO 92/00091. A significant improvement of the bead-based methods involves tagging each bead with a unique identifier tag, such as an oligonucleotide or electrophoretic tag, so as to facilitate identification of the amino acid sequence of each library member. These improved bead-based methods are described in International Publication No. WO 93/06121.

Another chemical synthesis screening method involves the synthesis of arrays of peptides (or peptidomimetics) on a surface wherein each unique peptide sequence is at a discrete, predefined location in the array. The identity of each library member is determined by its spatial location in the array. The locations in the array where binding interactions between a predetermined molecule and reactive library members occur is determined, thereby identifying the sequences of the reactive library members on the basis of spatial location. These methods are described in U.S. Pat. No. 5,143,854; International Publication Nos WO 90/15070 and WO 92/10092; Fodor et al. Science 251:767, 1991. Of particular use are display systems, which enable a nucleic acid to be linked to the polypeptide it expresses. Selection protocols for isolating desired members of large libraries are known in the art, as typified by phage display techniques. Such systems, in which diverse peptide sequences are displayed on the surface of filamentous bacteriophage, are useful for creating libraries of antibody fragments (and the nucleotide sequences that encoding them) for the in vitro selection and amplification of specific antibody fragments that bind a target antigen. The nucleotide sequences encoding the V_{H} and V_{L} regions are linked to gene fragments which encode leader signals that direct them to the periplasmic space of E. coli and the resultant antibody fragments are displayed on the surface of the bacteriophage, typically as fusions to bacteriophage coat proteins (e.g., pIII or pVIII). Alternatively, antibody fragments are displayed externally on lambda phage capsids (phage bodies). An advantage of phage-based display systems is that selected library members can be amplified simply by growing the phage containing the selected library member in bacterial cells. Furthermore, since the nucleotide sequence that encode the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (Kang et al., Proc. Natl. Acad. Sci. U.S.A., 88:4363, 1991; Clackson et al. Nature 352:624, 1991; Lowman et al. Biochemistry 30:10832, 1991; Burton et al., Proc. Natl. Acad. Sci. U.S.A., 88:10134, 1991; Hoogenboom et al. Nucleic Acids Res., 19.4133, 1991, incorporated herein by reference in their entirety). One particularly advantageous approach has been the use of scFv phage-libraries (Huston et al. Proc Natl Acad Sci U.S.A. 85:5879-5883, 1988; Chaudhary et al. Proc Natl Acad Sci U.S.A. 87:1066-1070, 1990; Clackson et al., 1991, (supra)). Various embodiments of scFv libraries displayed on bacteriophage coat proteins have been described. Further phage display approaches are also known, for example as described in International Publication Nos. WO 96/06213 and WO 92/01047 (Medical Research Council et al.) and International Publication No. WO 97/08320 (Morphosys) which are incorporated herein by reference. Other systems for generating libraries of polypeptides or nucleotides involve the use of cell-free enzymatic machinery for the in vitro synthesis of the library members. In one method, RNA molecules are selected by alternate rounds of selection against a target ligand and PCR amplification (Tuerk et al., Science 249:505, 1990; Ellington et al., Nature 346:818, 1990). A similar technique may be used to identify DNA sequences which bind to carbohydrate, polysaccharide, proteoglycan, glucosaminoglycans and the like. Similarly, in vitro translation can be used to synthesize polypeptides as a method for generating large libraries. These methods which generally comprise stabilized polysome complexes, are described further in International Publication No. WO88/08453. Alternative display systems which are not phage-based, such as those disclosed in International Publication Nos. WO 95/22625 and WO 95/11922 (Affymax) use the polysomes to display polypeptides for selection. These and all the foregoing documents also are incorporated herein by reference.

Some embodiments of the present invention contemplate using genetic or proteinaceous forms of IGFBP-2 or variants, derivatives, analogs or mimetics thereof or components of the EC/PC matrix or variants, analogs or mimetics thereof as agents of the present invention.

In some embodiments, the present invention contemplates the use of full-length IGFBP-2 or biologically active portions of those polypeptides. Typically, biologically active IGFBP-2 portions comprise a HB domain and are capable of binding to one or more EC/PC matrix components. A biologically active portion of a full-length polypeptide can be a polypeptide which is, for example, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, 120, 150, 300, or more amino acid residues in length.

The present invention also contemplates variant forms of the interacting molecules. “Variant” polypeptides include proteins derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is, they continue to possess the desired biological activity of the native protein (e.g., wound-treating activity). Such variants may result from, for example, genetic polymorphism or from human manipulation. Biologically active variants of a native IGFBP-2 polypeptide will have at least 40%, 50%, 60%, 70%, generally at least 75%, 80%, 85%, preferably about 90% to 95% or more, and more preferably about 98% or more sequence similarity with the amino acid sequence for the native protein as determined by sequence alignment programs described elsewhere herein using default parameters. A biologically active variant of a IGFBP-2 polypeptide may differ from that polypeptide generally by as much 100, 50 or 20 amino acid residues or suitably by as few as 1-15 amino acid residues, as few as 1-10, such as 6-10, as few as 5, as few as 4, 3, 2, or even 1 amino acid residue.

An IGFBP-2 polypeptide may be altered in various ways including amino acid substitutions, deletions, truncations, and insertions. Methods for such manipulations are generally known in the art. For example, amino acid sequence variants of an IGFBP-2 polypeptide can be prepared by mutations in the DNA. Methods for mutagenesis and nucleotide sequence alterations are well known in the art. See, for example, Kunkel (Proc Natl Acad Sci USA 82:488-492, 1985), Kunkel et al. (Methods in Enzymol 154:367-382, 1987), U.S. Pat. No. 4,873,192, Watson et al. (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al. Atlas of Protein Sequence and Structure (Natl Biomed Res Found, Washington, D.C., 1978). Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of IGFBP-2 polypeptides. Recursive ensemble mutagenesis (REM), a technique that enhances the frequency of functional mutants in the libraries, can be used in combination with the screening assays to identify IGFBP-2 polypeptide variants (Arkin et al., Proc Natl Acad Sci USA 89:7811-7815, 1992; Delgrave et al. Protein Engineering 6:327-331, 1993). Conservative substitutions, such as exchanging one amino acid with another having similar properties, may be desirable as discussed in more detail below.

Variant IGFBP-2 polypeptides may contain conservative amino acid substitutions at various locations along their sequence, as compared to the parent IGFBP-2 amino acid sequence. A “conservative amino acid substitution” is one in which the amino acid residue is replaced with an amino acid residue having a similar side chain. Families of amino acid residues having similar side chains have been defined in the art, which can be generally sub-classified as follows:
Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.
Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.
Charged: The residues are charged at physiological pH and, therefore, include amino acids having acidic or basic side chains (i.e., glutamic acid, aspartic acid, arginine, lysine and histidine).
Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.
Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterises certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al 1978 (supra); and by Gonnet et al. Science 256(5062):1443-1445, 1992), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behaviour.

Amino acid residues can be further sub-classified as cyclic or noncyclic, and aromatic or nonaromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always nonaromatic. Dependent on their structural properties, amino acid residues may fall in two or more classes. For the naturally-occurring protein amino acids, sub-classification according to this scheme is presented in the Table 2.

TABLE 2
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional IGFBP-2 polypeptide can readily be determined by assaying its activity. Conservative substitutions are shown in Table 3 below under the heading of exemplary substitutions. More preferred substitutions are shown under the heading of preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site; or (c) the bulk of the side chain. After the substitutions are introduced, the variants are screened for biological activity.

TABLE 3
Exemplary and Preferred Amino Acid Substitutions

| | EXEMPLARY | PREFERRED |
|---|---|---|
| Original Residue | SUBSTITUTIONS | SUBSTITUTIONS |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, Biochemistry third edition, Wm. C. Brown Publishers, 1993.

Thus, a predicted non-essential amino acid residue in a IGFBP-2 polypeptide is typically replaced with another amino acid residue from the same side chain family. Alternatively, mutations can be introduced randomly along all or part of a IGFBP-2 polynucleotide coding sequence, such as by saturation mutagenesis, and the resultant mutants can be screened for an activity of the parent polypeptide to identify mutants which retain that activity. Following mutagenesis of the coding sequences, the encoded peptide can be expressed recombinantly and the activity of the peptide can be determined.

Accordingly, the present invention also contemplates variants of the naturally-occurring IGFBP-2 polypeptide sequences or their biologically-active fragments, wherein the variants are distinguished from the naturally-occurring sequence by the addition, deletion, or substitution of one or more amino acid residues. In general, variants will display at least about 30, 40, 50, 55, 60, 65, 70, 75, 80, 85, 90, 91, 92, 93, 94, 95, 96, 97, 98, 99% similarity to a parent IGFBP-2 polypeptide sequence as, for example, set forth in any one of SEQ ID NO: 2. Desirably, variants will have at least 30, 40, 50, 55, 60, 65, 70, 75, 80, 85, 90, 91, 92, 93, 94, 95, 96, 97, 98, 99% sequence identity to a parent IGFBP-2 polypeptide sequence as, for example, set forth in any one of SEQ ID NO: 2. Moreover, sequences differing from the native or parent sequences by the addition, deletion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100 or more amino acids but which retain the properties of the parent IGFBP-2 polypeptide are contemplated. IGFBP-2 polypeptides also include polypeptides that are encoded by polynucleotides that hybridize under stringency conditions as defined herein, especially high stringency conditions, to IGFBP-2 polynucleotide sequences, or the non-coding strand thereof.

In some embodiments, variant polypeptides differ from an IGFBP-2 sequence by at least one but by less than 50, 40, 30, 20, 15, 10, 8, 6, 5, 4, 3 or 2 amino acid residue(s). In another, variant polypeptides differ from the corresponding sequence in any one of SEQ ID NO: 2 by at least 1% but less than 20%, 15%, 10% or 5% of the residues. (If this comparison requires alignment the sequences should be aligned for maximum similarity. “Looped” out sequences from deletions or insertions, or mismatches, are considered differences.) The differences are, suitably, differences or changes at a non-essential residue or a conservative substitution.

A “non-essential” amino acid residue is a residue that can be altered from the wild-type sequence of an embodiment polypeptide without abolishing or substantially altering one or more of its activities. Suitably, the alteration does not substantially alter one of these activities, for example, the activity is at least 20%, 40%, 60%, 70% or 80% of wild-type. An “essential” amino acid residue is a residue that, when altered from the wild-type sequence of an IGFBP-2 polypeptide of the invention, results in abolition of an activity of the parent molecule such that less than 20% of the wild-type activity is present.

In other embodiments, a variant polypeptide includes an amino acid sequence having at least about 50%, 55%, 60%, 65%, 70%, 75%, 80%, 85%, 90%, 91%, 92%, 93%, 94% 95%, 96%, 97%, 98% or more similarity to a corresponding sequence of a IGFBP-2 polypeptide as, for example, set forth in any one of SEQ ID NO: 2, and has the activity of that IGFBP-2 polypeptide.

IGFBP-2 polypeptides may be prepared by any suitable procedure known to those of skill in the art. For example, the polypeptides may be prepared by a procedure including the steps of: (a) preparing a chimeric construct comprising a nucleotide sequence that encodes at least a portion of a IGFBP-2 polynucleotide and that is operably linked to a regulatory element; (b) introducing the chimeric construct into a host cell; (c) culturing the host cell to express the IGFBP-2 polypeptide; and (d) isolating the IGFBP-2 polypeptide from the host cell. In illustrative examples, the nucleotide sequence encodes at least a portion of the sequence set forth in any one of SEQ ID NO: 2, or a variant thereof. Recombinant IGFBP-2 polypeptides can be conveniently prepared using standard protocols as described for example in Sambrook, et al. 1989 (supra), in particular Sections 16 and 17; Ausubel et al. Current Protocols in Molecular Biology, 5^{th} Edition, John Wiley & Sons, Inc, N.Y., 2002, in particular Chapters 10 and 16; and Coligan et al. Current Protocols In Protein Science (John Wiley & Sons, Inc. 1995-1997), in particular Chapters 1, 5 and 6. Alternatively, the IGFBP-2 polypeptides may be synthesised by chemical synthesis, e.g., using solution synthesis or solid phase synthesis as described, for example, Atherton et al., (supra) and in Roberge et al. (Science 269:202, 1995).

Another useful group of compounds are functional derivatives, analogs and mimics (mimetics) of the subject agents. In accordance with the present invention, these molecules retain the ability to bind to IGFBP-2 or EC/PC matrix components and may also possess additional characteristics which enhance their therapeutic efficacy, such as a longer half life in vivo or alternatively which are, for example, readily synthesized or readily taken up by cells. A peptide mimetic or mimic has some chemical similarity to the parent molecule e.g., IGFBP-2, but agonizes its activity. A peptide mimic may be a peptide-containing molecule which mimics elements of protein secondary structure (as described for example in Johnson et al. “Peptide Turn Mimetics” in Biotechnology and Pharmacy, Pezzuto et al. Eds., Chapman and Hall, New York, 1993) Peptide or non-peptide mimetics may be useful, for example, in regulating IGFBP-2 dependent pathways in the treatment of cancer. Non-peptide “small molecules” are often preferred for many in vivo pharmaceutical applications and accordingly mimetics may be designed for pharmaceutical use. Mimetic design, synthesis and testing is generally used to avoid randomly screening large numbers of molecules for a particular property, particularly where a lead compound has already been identified. As a first step, residues critical for inhibiting target activity are identified and this framework used as a pharmacophore. The structure may then be modeled using computational and other analyses. Alternatively, the three dimensional structure of the inhibitor may be known in which case further agents may be designed along the same lines.

The goal of rational drug design is to produce structural analogs of biologically active molecules of interest or of small molecules with which they interact (e.g. agonists, antagonists, inhibitors or enhancers) in order to fashion drugs which are, for example, more active or stable forms of the molecules, or which, e.g. enhance or interfere with the function of a molecule in vivo. See, e.g. Hodgson (Bio/Technology 9:19-21, 1991). In one approach, one first determines the three-dimensional structure of a protein of interest by x-ray crystallography, by computer modeling or most typically, by a combination of approaches. Useful information regarding the structure of a polypeptide may also be gained by modeling based on the structure of homologous proteins. An example of rational drug design is the development of HIV protease inhibitors (Erickson et al. Science 249:527-533, 1990). In addition, target molecules may be analyzed by an alanine scan (Wells Methods Enzymol 202:2699-2705, 1991). In this technique, an amino acid residue is replaced by Ala and its effect on the peptide's activity is determined. Each of the amino acid residues of the peptide is analyzed in this manner to determine the important regions of the peptide.

It is also possible to isolate a target-specific antibody, selected by a functional assay and then to solve its crystal structure. In principle, this approach yields a pharmacophore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacophore.

In other aspects of the present invention, a genetic approach may be taken to regulating IGFBP-2 dependent pathways through the HBD:IBD interation. Antisense oligonucleotide sequences, for example, are useful in silencing IGFBP-2 transcripts. Furthermore, co-suppression and molecules to induce iRNA or siRNA may also be conveniently employed. Antisense, or sense molecule may be directly administered in suitable compositions or administered in polynucleotide vectors. Oligonucleotides comprising morpholino nucleotide derivatives and phosphorodiamidate linkages are also contemplated as described in Summerton et al., Antisense and Nucleic Acid Drug Development 7:187-195, 1997. The functions of DNA to be interfered with can include replication and transcription. The functions of RNA to be interfered with can include functions such as translocation, translation, splicing, catalytic activity or complex formation. In either case, the end result is reduced activity of the target molecule.

Other nucleic acid or oligonucleotide molecules which may be used in accordance with the present invention are antisense oligonucleotides, ribozymes, external guide sequences, primers, probes and other oligomeric compounds which bind to at least a part of the target nucleic acid molecule. Alternatively 3′ or 5′ regions of the target nucleic acid may be targeted, such as the 5′ untranslated region or the cap sire of an mRNA. These molecules may be administered in the form of single or double stranded, circular or hairpin molecules where they directly or indirectly effect modification of the target nucleic acid.

RNA and DNA aptamers can substitute for monoclonal antibodies in various applications (Jayasena, Clin. Chem., 45(9):1628-1650, 1999; Morris et al., Proc. Natl. Acad. Sci., USA, 95(6):2902-2907, 1998). Aptamers are nucleic acid molecules having specific binding affinity to non-nucleic acid or nucleic acid molecules through interactions other than classic Watson-Crick base pairing. Aptamers are described, for example, in U.S. Pat. Nos. 5,475,096; 5,270,163; 5,589,332; 5,589,332; and 5,741,679.

An increasing number of DNA and RNA aptamers that recognize their non-nucleic acid targets have been developed by SELEX and have been characterized (Gold et al., Annu. Rev. Biochem., 64:763-797.1995; Bacher et al., Drug Discovery Today, 3(6):265-273, 1998).

Following identification of a suitable agent, the agent or a composition comprising the agent is administered to individuals in need of treatment or regenerative therapy.

As described in more detail below, in some embodiments, the agents or compositions comprising the agents are administered directly to the tumor site. In some embodiments, direct delivery of the agent involves administering the agent attached to or otherwise associated with a medical or other device, tissue or composition. The agents and compositions can be formulated into a wide variety of carriers. For example, the agents are formulated together with carriers such as microparticles, gels, bioactive foams, synthetic skin preparations, liquids and creams. Microparticles may for example be microspheres, microcapsules, liposomes and the like adapted for slow release of agents over time. Such particles are conveniently biodegradable or biocompatible.

In other embodiments, the compositions are administered indirectly by any convenient means such as without limitation orally, infusively, intravenously, respiratorally, intratracheally, intracerebrally, nasopharyngeally, intranasally, intraocularly, intracranially, intraperitoneal, intramuscular, subcutaneously, intradermally or by suppository routes, i.v. drip/patch or implant. In some embodiments, agents are attached to targeting molecules to facilitate delivery to the tumor site, for example antibody targeting molecules may be employed. In other embodiments, the agents are delivered in genetic form either directly to the tumor site or indirectly, and in the form, for example, of a DNA vector such as a viral or non-viral expression vector expressing proteinaceous forms of the agent, a transformed, transfected or otherwise modified cell expressing and secreting proteinaceous forms of the agent, sense, antisense or inhibitory RNA (iRNA) molecules. By whatever route or combination of routes, the active agent is administered in a therapeutically or prophylactically effective amount.

The polypeptides, nucleic acids, peptides, chemical analogs or mimetics of the present invention can be formulated in pharmaceutical compositions which are prepared according to conventional pharmaceutical compounding techniques. See, for example, Remington's Pharmaceutical Sciences, 18^{th} Ed. (1990, Mack Publishing, Company, Easton, Pa., U.S.A.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. intravenous, oral or parenteral.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract. See for example, International Patent Publication No. WO 96/11698.

For parenteral administration, the compound may dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like.

The actual amount of active agent administered and the rate and time-course of administration will depend various factors. Prescription of treatment, e.g. decisions on dosage, timing, etc. is within the responsibility of general practitioners or specialists and typically takes account the condition of the individual patient, the site of delivery, the method of administration and other factors known to practitioners. Examples of techniques and protocols can be found in Remington's 1990 (supra).

The pharmaceutical composition is contemplated to exhibit therapeutic activity when administered in an amount which depends on the particular case. The variation depends, for example, on the human or animal and the inhibitory agent chosen. A broad range of doses may be applicable. Considering a patient, for example, from about 0.1 ng, 0.2 ng, 0.3 ng, 0.4 ng, 0.5 ng, 0.6 ng, 0.7 ng, 0.8 ng. 0.9 ng, or 0.1 mg, 0.2 mg, 0.3 mg, 0.4 mg, 0.5 mg, 0.6 mg, 0.7 mg, 0.8 mg. 0.9 mg to about 1 to 10 mg or from 5 to 50 mg of inhibitory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The agents may be administered in a convenient manner such as by the oral, intravenous (where water soluble), intraperitoneal, intramuscular, subcutaneous, intradermal or suppository routes or implanting (e.g. using slow release molecules). The agent or composition comprising the agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulfate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Alternatively, genetic targeting therapies may be used to deliver the active agent more specifically to certain types of cell, by the use of targeting systems such as antibodies or cell specific ligands. Targeting may be desirable for a variety of reasons, e.g. if the agent is unacceptably toxic or if it would otherwise require too high a dosage or if it would not otherwise be able to enter the target cells.

Gene therapy would be carried out according to generally accepted methods, for example, as described by Friedman (In: Therapy for Genetic Disease, T. Friedman, Ed., Oxford University Press, pp. 105-121, 1991) or Culver (Gene Therapy: A Primer for Physicians, 2^{nd} Ed., Mary Ann Liebert, 1996). Suitable vectors are known, such as disclosed in U.S. Pat. No. 5,252,479, International Patent Publication No. WO 93/07282 and U.S. Pat. No. 5,691,198. Gene transfer systems known in the art may be useful in the practice of the gene therapy methods of the present invention. These include viral and non-viral transfer methods. A number of viruses have been used as gene transfer vectors or as the basis for preparing gene transfer vectors, including papovaviruses (e.g. SV40, Madzak et al., J. Gen. Virol., 73:1533-1536, 1992), adenovirus (Berkner, Curr. Top. Microbiol. Immunol., 158:39-66, 1992; Berkner et al., BioTechniques, 6:616-629, 1988; Gorziglia et al., J. Virol., 66:4407-4412, 1992; Quantin et al., Proc. Natl. Acad. Sci. USA, 89:2581-2584, 1992; Rosenfeld et al., Cell, 68.143-155, 1992; Wilkinson et al., Nucleic Acids Res., 20:2233-2239, 1992; Stratford-Perricaudet et al., Hum. Gene Ther., 1:241-256, 1990; Schneider et al., Nature Genetics, 18:180-183, 1998), vaccinia virus (Moss, Curr. Top. Microbiol. Immunol., 158:25-38, 1992; Moss, Proc. Natl. Acad. Sci. USA, 93:11341-11348, 1996), adeno-associated virus (Muzyczka, Curr. Top. Microbiol. Immunol., 158:97-129, 1992; Ohi et al., Gene, 89:279-282, 1990; Russell et al., Nature Genetics, 18:323-328, 1998), herpesviruses including HSV and EBV (Margolskee, Curr. Top., Microbiol. Immunol., 158:67-95, 1992; Johnson et al., J. Virol., 66:2952-2965, 1992; Fink et al., Hum. Gene Ther., 3:11-19, 1992; Breakefield et al., Mol. Neurobiol., 1:339-371, 1987; Freese et al., Biochem. Pharmacol., 40:2189-2199, 1990; Fink et al., Ann. Rev. Neurosci., 19:265-287, 1996), lentiviruses (Naldini et al., Science, 272:263-267, 1996), Sindbis and Semliki Forest virus (Berglund et al., Biotechnology, 11.916-920, 1993) and retroviruses of avian (Bandyopadhyay and Temin, Mol. Cell. Biol., 4:749-754, 1984; Petropoulos et al., J. Viol., 66.3391-3397, 1992), murine (Miller, Curr. Top. Microbiol. Immunol., 158:1-24, 1992; Miller et al., Mol. Cell. Biol., 5:431-437, 1985; Sorge et al., Mol. Cell. Biol., 4:1730-1737, 1984; Mann et al., J. Virol., 54:401-407, 1985; Miller et al., J. Virol., 62:4337-4345, 1988) and human (Shimada et al., J. Clin. Invest., 88:1043-1047, 1991; Helseth et al., J. Virol, 64:2416-2420, 1990; Page et al., J. Virol, 64:5270-5276, 1990; Buchschacher et al., J. Virol., 66:2731-2739, 1982) origin.

Instead of administering these agents directly, they could be produced in the target cell, e.g. in a viral vector such as described above or in a cell based delivery system such as described in U.S. Pat. No. 5,550,050 and International Patent Publication Nos. WO 92/19195, WO 94/25503, WO 95/01203, WO 95/05452, WO 96/02286, WO 96/02646, WO 96/40871, WO 96/40959 and WO 97/12635. The vector could be targeted to the target skin cells or expression of expression products could be limited to specific cells, stages of development or cell cycle stages. The cell based delivery system is designed to be implanted in a patient's body at the tumor site. Alternatively, the agents could be administered in a precursor form for conversion to the active form by an activating agent produced in, or targeted to, the cells to be treated. See, for example, European Patent Application No. 0 425 731A and International Patent Publication No. WO 90/07936.

The present invention further provides a method of assessing the susceptibility of a subject to developing a metastatic form of cancer comprising screening for mutations in the HBD of IGFBP wherein the presence of a functional HBD is indicative that the subject is susceptible to metastatic cancer.

A wide range of mutation detection screening methods are available as would be known to those skilled in the art. Any method which allows an accurate comparison between a test and control nucleic acid sequence may be employed. Scanning methods include sequencing, denaturing gradient gel electrophoresis (DGGE), single-stranded conformational polymorphism (SSCP and rSSCP, REF-SSCP), chemical cleavage methods such as CCM, ECM, DHPLC and MALDI-TOF MS and DNA chip technology. Specific methods to screen for pre-determined mutations include allele specific oligonucleotides (ASO), allele specific amplification, competitive oligonucleotide priming, oligonucleotide ligation assay, base-specific primer extension, dot blot assays and RFLP-PCR. The strengths and weaknesses of these and further approaches are reviewed in Sambrook, Chapter 13, Molecular Cloning, 2001.

Useful diagnostic techniques to detect aberrations in the MGMT gene include but are not limited to fluorescent in situ hybridization (FISH), direct DNA sequencing, PFGE analysis, Southern blot analysis, single-stranded conformational analysis (SSCA), Rnase protection assay, allele-specific oligonucleotide (ASO hybridization), dot blot analysis and PCR-SSCP (see below).

There are several methods that can be used to detect DNA sequence variation. Direct DNA sequencing, either manual sequencing or automated fluorescent sequencing, can detect sequence variation. Another approach is the single-stranded conformation polymorphism assay (SSCP) (Orita et al., Proc. Nat. Acad. Sci. USA, 86:2776-2770, 1989). This method can be optimized to detect most DNA sequence variation. The increased throughput possible with SSCP makes it an attractive, viable alternative to direct sequencing for mutation detection on a research basis. The fragments which have shifted mobility on SSCP gels are then sequenced to determine the exact nature of the DNA sequence variation. Other approaches based on the detection of mismatches between the two complementary DNA strands include clamped denaturing gel electrophoresis (CDGE) (Sheffield et al., Am. J. Hum. Genet., 49:699-706, 1991), heteroduplex analysis (HA) (White et al., Genomics, 12:301-306, 1992) and chemical mismatch cleavage (CMC) (Grompe et al., Proc. Natl. Acad. Sci. USA, 86:5855-5892, 1989). Other methods which might detect mutations in regulatory regions or which might comprise large deletions, duplications or insertions include the protein truncation assay or the asymmetric assay. A review of methods of detecting DNA sequence variation can be found in Grompe (Proc. Natl. Acad. Sci. USA, 86:5855-5892, 1993). Once a mutation is known, an allele specific detection approach such as allele specific oligonucleotide (ASO) hybridization can be utilized to rapidly screen large numbers of other samples for that same mutation. Such a technique can utilize probes which are labeled with gold nanoparticles to yield a visual color result (Elghanian et al., Science, 277.1078-1081, 1997).

The present invention is further described by the following non-limiting Examples.

### Example 1

### Material & Methods

### Reagents

Human-IGF-I was from Kabi Pharmacia Upjohn, Peptide Hormones Sweden. ¹²⁵1-IGF-I (˜2000 Ci/mmol) was bought from Amersham (North Ryde, NSW, Australia). The full-length human IGFBP-2 cDNA was kindly provided by Dr David Clemmons (University of North Carolina, Chapel Hill, N.C.). Aggrecan, fibronectin and heparin were kindly supplied by Dr. Amanda Fosang (Arthritis Research Unit, MCRI, Royal Children's Hospital, Parkville, Victoria). Human vitronectin was purchased from Promega (Promega Corporation, Annadale, NSW, Australia). Bovine Collagen type IV and mouse Laminin were a gift from Dr Shireen Lamande (Cell & Matrix Research Unit, MCRI, Royal Children's Hospital, Parkville Victoria).

### Cell Culture

The human neuroblastoma SK-N-SHEP (SHEP) cell line was supplied by Dr. Eva Feldman (Dept of Neurology, University of Michigan, Ann Arbor, Mich.). SHEP cells express very low amounts of IGF-I, IGF-II, IGF-I receptors and do not express IGFBP-2 (van Golen et al. Cell Death Differ 7:654-665, 2000, Russo et al. Brain Research 1009:40-53, 2004). SHEP cells were grown in DMEM/10% FCS (Trace Biosciences, Castle Hill, NSW) or as indicated.

### Rat Olfactory Bulb Cell Membranes

Rat olfactory bulbs (OB), fresh or from a frozen stock stored at −70° C., were resuspended in ice cold buffer (10 mM Tris/HCl, 2 mM PMSF, 1 TIU/ml of Aprotinin) and mechanically disaggregated through 19 gauge and 23 gauge needle syringes. The tissue suspension was centrifuged at 500 rpm for 5 min at 4° C. and the supernatant was centrifuged at 14,000 rpm for 1 hour at 4° C. The pellet, called the starting material (SM), was resuspended in ice cold 10 mM Tris/HCl pH 7.4. Aliquots of SM were adjusted to a total protein concentration of 100 μg/80 μl, followed by addition of 0.1% BSA and storage at −20° C. until required. The animal procedures performed to obtain rat brain olfactory bulbs were approved by the Royal Children's Hospital Animal Ethic Committee.

### ¹²⁵I-IGF-I Binding and Cross-Linking to Rat Brain Olfactory Bulb (OB) Membranes

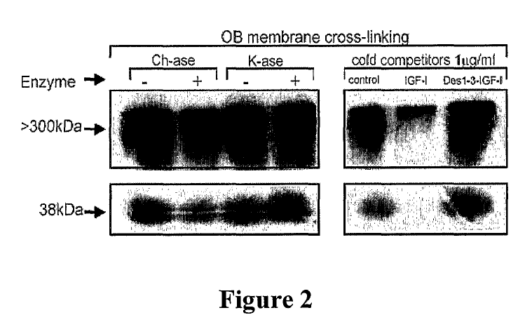

Rat OB membranes (100 μg, SM) were incubated in the absence (not-treated=NT) or presence of NaCl (200-500 mM) or EDTA (20-30 mM) for 16 hours at 4° C. Alternatively, OB membrane (SM) were treated with Chondroitin ABC lyase (Proteus vulgaris) and keratanase (Pseudomonas sp.) (Seikagaku Kogyo, Japan) to digest glycosaminoglycan (GAG) chains (Firth et al., 2002 (supra)). OB membranes (SM, NT, NaCl or EDTA or GAG-enzyme treated) were then incubated with ¹²⁵I-IGF-I (˜40,000 cpm/tube) in the presence or absence of 1 μg/ml IGF-I or des(1-3)-IGF-I, as indicated in FIG. 2, for 2 hours at 37° C. with rotation. Samples were cross-linked with 1 mM DSS for 15 min at 4° C., separated by denaturing 3-16% gradient SDS-PAGE under reducing conditions, and the dried gel exposed to X-ray film for 5-10 days.

### Example 2

### Production of Modified IGFBP-2

A family of six high affinity IGF-binding proteins (IGFBP-1 through IGFBP-6) coordinate and regulate IGF's biological activity in several ways: 1) transport IGF in plasma and control its diffusion and efflux from the vascular space; 2) increase the half-life and regulate clearance of the IGFs; 3) provide specific binding sites for the IGFs in the extracellular and pericellular space; 4) modulate, inhibit or facilitate interaction of IGFs with their receptors (Rajaram et al. Endocrine Reviews 18:801-831, 1997, Jones et al., Endocrine Reviews 16:3-34, 1995, Bach et al., Diabetes Review 3:38-61, 1995, Russo et al. 1997 (supra), Firth et al., 2002 (supra)) IGFBP biological activity is regulated by post-translational modifications such as glycosylation and phosphorylation (Jones et al., 1995 (supra)) and or differential localization of the IGFBPs in the pericellular and extracellular space (Jones et al., 1995 (supra), Russo et al. 1997 (supra), Firth et al., 2002 (supra), Russo et al. 1999 (supra), Parker et al. Journal of Biological Chemistry 271:13523-13529, 1996, Parker et al. Molecular Biology of the Cell 9:2383-2392, 1998, Rees et al., Journal of Cellular Biochemistry 71.375-381, 1998). It is therefore hypothesised that IGFBPs, in addition to stabilizing and regulating levels of diffusible IGFs, might regulate IGF-I cellular responses by facilitating receptor targeting of IGF-I or modulating IGF-I bioavailability in the pericellular space (Jones et al., 1995 (supra), Firth et al., 2002 (supra)).

The effects of IGFBPs are further regulated by the presence of specific IGFBP proteases, which cleave the binding proteins, generating fragments with reduced or no binding affinity for the IGFs (Jones et al., 1995 (supra), Firth et al., 2002 (supra), Russo et al. 1999 (supra), Binoux et al. Contemporary Endocrinology: The IGF system pp 281-313, 1999). Some IGFBPs, including IGFBP-2 and -3, can induce direct cellular effects independent of the IGFs (Firth et al., 2002 (supra), Oh et al. Journal of Biological Chemistry 268:14964-14971, 1993, Oh et al. Journal of Biological Chemistry 268:26045-26048, 1993, Yamanaka et al. Endocrinology 140:1319-1328, 1999, Schutt et al. J Mol Endocrinol 32:859-68, 2004). IGFBP-3, similar to IGFBP-5, contains sequences with the potential for nuclear localization (Radulescu Trends in Biochemical Science 19:278, 1994, Schedlich et al. Journal of Biological Chemistry 273:18347-18352, 1998) and detection of IGFBP-3 in the nuclei of dividing cells, as reported by several investigators (Schedlich et al. 1998 (supra), Wraight et al. Journal of Investigative Dermatology 111:239-242, 1998), strongly suggests a role for IGFBP-3 in gene regulation. More recently, peri-nuclear or nuclear localisation has also been reported for IGFBP-2 (Hoeflich et al. Biochem Biophys Res Commun 324:705-10, 2004), however the role of IGFBP-2 in this cellular compartment is yet to be determined.

The HBD of IGFBP-2 at ₁₇₉PKKLRP₁₈₄ was mutated to ₁₇₉PNNLAP₁₈₄ by PCR based mutagenesis. A “mutagenic cassette” was generated by routine procedures combining the external primers 5′-₅₈₉GAAGGAGGCCTGGTGGAGAACC₆₁₀-3′ (forward, A:SEQ ID NO:3) and 5′-₁₀₁₅CCGGGAAGCTGATCCAGGGAG₉₉₅-3′ (reverse, D:SEQ ID NO:4) with the internal mutagenic primers 5′-₇₅₈GCCTGGAGGAGCCCAACAACCTGGCACCACCCCCTGCCAG₇₉₇-3′ (forward, B:SEQ ID NO:5) and 5′-₇₉₇ CTGGCAGGGGGTGGTGCCAGGTTGTTGGGCTCCTCCAGGC₇₅₈-3′ (reverse, C:SEQ ID NO:6). The overlapping PCR products A-C and B-D were hybridized, extended, and the mutated cDNA amplified using A and D primers.

The ₂₆₅RGD₂₆₇ of IGFBP-2 was mutated to ₂₆₅RGE₂₆₇ by using a forward primer 5′-₇₈₇ACCATCCGGGGGGAACCCGAGTG₈₀₉-3′ (SEQ ID NO:7) (introducing the point mutation) and a reverse primer 5′-CAACCGGTₛₜₒₚCTACTGCATCCGCTGGGTGTG-3′ (SEQ ID NO:8) (introducing a stop codon and an AgeI restriction site) generating, a 88 bp PCR product (reverse mutagenic megaprimer). The reverse mutagenic megaprimer was then used in combination with the forward primer 5′-CTCGAG1ATGCTGCCGAGAGTCGGCTGC21-3′ (SEQ ID NO:9) (introducing a start codon and a Xho-I restriction site) to amplify mutated (RGE) full length IGFBP-2 cDNA. The mutated full-length IGFBP-2 cDNA was then subcloned into the Xho-I and Age-I restriction sites of the pcDNA3.1/V5-His A mammalian expression vector (Invitrogen, Karlruhe, Germany) to express an untagged RG²⁶⁷D/E-IGFBP-2 (pcDNA3.1-RGE²⁶⁷ IGFBP-2). DNA sequencing of the full-length IGFBP-2 mutated clones was performed to verify that the required mutations were present in the HBD and RGD motifs and that no other alterations were introduced in the IGFBP-2 cDNA clones.

As described earlier the IGFBP-2 HBD at ₁₇₉PKKLRP₁₈₄ was mutated to ₁₇₉PNNLAP₁₈₄ by PCR based mutagenesis. This heparin binding domain also contains a site sensitive to proteolysis (₁₇₉PKKLRP₁₈₄), as described by Ho et al. (Endocrinology, 138(9):3811-3818, 1997). Thus the HBD mutant (₁₇₉PNNLAP₁₈₄) is potentially a proteolytic-cleavage resistant mutant and therefore it has alternatively named the HBD mutant as PCS3.

A larger proteolytic domain, sensitive to a number of proteases (i.e. Kallikrein. PAPP-A, etc), is present upstream to the IGFBP-2 HBD. Five potential cleavage sites have been identified in this by amino-terminal sequencing of the isolated IGFBP-2 fragments. Therefore four of these sites were mutated by using the QuikChange site directed mutagenesis kit from Stratagene. These mutations were introduced in both the WT-IGFBP-2 generating a PCS-IGFBP-2 mutant with 4 sites mutated (PCS4) and into the HBD-IGFBP-2 mutant generating a PCS-IGFBP-2 mutant with 7 sites mutated (PCS7) the mutagenic primer (45 bases, Tm 78C) used was as follows: 5′CTGAGCAGCACGCGGCGATGGGCGGGGGTGCGCATCACCTTG3′(SEQ ID NO:10) the complementary reverse primer was 5′CAAGGTGATGCGCACCCCCGCCCATCGCCGCGTGCTGCTCAG3′ (SEQ ID NO:11). Mutagenesis was according to the manufacturer's specifications (Stratagene). DNA sequencing of the full-length IGFBP-2 mutated PCS clones was performed to verify that the required mutations were present in the PCS domains for PCS4 and 7 and that no other alterations were introduced in the IGFBP-2 cDNA PCS clones.

### Example 3

### Mammalian Expression Vector Provides Stable Transfection of Native and Mutant IGFBP-2 in SHEP Cells

Native (WT) and mutant HBD hIGFBP-2 cDNAs were sub-cloned into the HindIII/BglII digested mammalian expression plasmid pCMV-int (Hoeflich et al. 1999 (supra)).

The pCMV-int-_{WT}IGFBP-2 or pCMV-int-_{HBD}IGFBP-2 and pSV2-Neo (the latter used to confer resistance to the selecting agent Neomycin-G418 at 300 μg/ml) or the pcDNA3.1-RGE²⁶⁷ IGFBP-2 construct were then transfected into SK-N-SHEP cells, using the Profection Calcium Phosphate Mammalian Transfection kit (Promega Corporation, Annadale, NSW, Australia), according to the manufacturer's specifications. Stable WT, HBD and RGE IGFBP-2 transfectant clones were isolated following G418 selection.

IGFBP-2 in conditioned medium was determined by Western ligand blotting (WLB) (Hossenlopp et al. Analytical Biochemistry 154:138-143, 1986) and immunoblotting (Russo et al. 1997 (supra)). IGFBP-2 levels were quantified by the R&D IGFBP-2 ELISA (R&D System, Minneapolis, Minn., USA) or an in-house IGFBP-2 RIA (Elmlinger et al. Growth Regul 6:152-157, 1996). An average of 15-20 clones were isolated for each of the transfectants. The isolated clones for the HBD or RGE-IGFBP-2 mutant were matched as closely as possible to those expressing similar level of WT-IGFBP-2.

PCS mutant IGFBP-2 were purified by IGF-I affinity chromatography as previously described by Ho et al. 1997 (supra). Fractions were analysed by WLB (125I-IGF-I) and immunoblotting and quantified by the R&D IGFBP-2 ELISA or RIA as described earlier.

Native or mutant IGFBP-2 were purified by IGF-I affinity chromatography as previously described by Ho et al. 1997 (supra). Fractions were analysed by WLB and immunoblotting and quantified by the R&D IGFBP-2 ELISA or RIA as above.

### Example 4

### Native and mutant IGFBP-2 retain binding affinity for IGF-I/II

The impact of these exemplary mutations in IGFBP-2 were tested to see if they affected IGF binding affinity. Native, HBD and RGD-IGFBP-2 (all 2.5 ng) were incubated with either ¹²⁵I-IGF-I or -II (15,000 cpm) in the presence of increasing concentrations of unlabeled IGF-I or -II (0.0025-0.15 nM). Binding was for 2 h at room temperature in 100 μl binding buffer [BB; 50 mM sodium phosphate pH 7.4, 0.1 M NaCl, 0.05% (w/v) NaN3, 0.2% fatty acid free BSA (Sigma, Steinheim, Germany), 0.1% (v/v) Triton X-100]. Bound and free ¹²⁵I-IGF-I or -II were separated by adding 0.1 ml ice-cold antibody solution [1:4000 polyclonal anti-IGFBP-2 antibody, 0.05 mg/ml rabbit IgG (Sigma, Steinheim, Germany) in BB] for 16 hours at 4° C. followed by precipitation for 1 hour at 4° C. with 500 μl of an anti-rabbit IgG antibody solution [1:300 sheep anti-rabbit IgG (Sigma, Steinheim, Germany) in 4% Polyethylene glycol (PEG 6000)]. Antibody complexes were precipitated by centrifugation (4° C., 3500×g, 15 min) and washed once with 1 ml ice-cold water. Bound radioactivity in precipitates was quantified in a γ-counter. Each point was measured in quadruplicate. The Sigma Plot 8.0 graphic program (Jandel Scientific, San Rafael, Calif., USA) was used to calculate binding affinities using a one binding-site hyperbolic fit.

### Example 5

### Native and Mutant-IGFBP-2 Binding to ECM Components

A solid phase binding assay (Russo et al. 1997 (supra)) was used to determine whether the mutations introduced into IGFBP-2 affected its binding to aggrecan, heparin, laminin, fibronectin, collagen type IV (all at 500 ng/200 μl) (Russo et al. 1997 (supra), McCaig et al. J Cell Sci 115:4293-4303, 2002) or vitronectin (300 ng/200 μl) (Kricker et al. Endocrinology 144:2807-2815, 2003). Following removal of unbound IGFBP-2, wells were incubated for 16 hours at 4° C. with ¹²⁵I-IGF-I (1.5×10⁴−3×10⁴ cpm) in the presence (NS=non-specific binding) or absence (TB=total binding) of an excess of unlabelled IGF-I (1 μg/ml). All wells were then washed four times with binding buffer and bound radioactivity was measured in a γ-counter. In the absence of native or mutant IGFBP-2, ¹²⁵I-IGF-I binding to all of the coated substrates was undetectable (data not shown). Each point was measured in triplicate. Non specific binding was less than 1.5% of the added radioactivity for all these conditions.

### Example 6

### SHEP Cell Proliferation Following Addition of Native or Modified IGFBP-2

SHEP cells (3.0×10⁴) were seeded in 96 well-plates and cultured in 200 μl of DMEM/10% FCS. After 12 hours, cells were washed and media changed to 200 μl serum free media (SFM) containing 1 μCi ³H-thymidine with or without IGF-I (100 ng/ml) and/or affinity purified WT, HBD or RGE-IGFBP-2 (800 ng/ml). After 36 hours, cells were harvested and DNA was immobilized onto a nitrocellulose filter/membrane by the Harvester 96 (Tomtec, Hamden, USA). Radioactivity incorporated into DNA was counted with a beta-counter (MicroBeta 1450, Wallac, Milton Keynes, UK). Samples run in quadruplicate.

### Example 7

### Effects of Over-Expression of Native or Mutant IGFBP-2 on SHEP Cell Proliferation

Two clones for each of the SHEP cells over-expressing similar level (931.8±75.4 ng/ml at 72 hours; 1193.0±136.6 ng/ml at 96 hours) of either WT, HBD or RGE-IGFBP-2 were grown in DMEM/10% FCS and treated as follows:

SHEP cells (4.5×10⁴ cells) were seeded in triplicate in T-25 flasks and maintained for up to 4 days without a media change. Cells were then trypsinised, stained with Trypan-blue, and counted using a haemocytometer (Neubauer-chamber) to ascertain cell numbers. Samples run in triplicate.

### Example 8

### Cell Migration and Invasion Assays

Cell migration/motility was measured using Falcon cell culture PET inserts with a pore-size of 8 μm in a 24 well format (Falcon No 353097, Le Pont de Claix, France). Cells were detached with 5 mM EDTA in PBS over 30 min at 37° C., harvested by centrifugation (200 x g, RT, 5° C.), washed 3 times with SFM containing 1.5% BSA. Cells (50×10⁴/500 μl) were then seeded in SFM/1.5% BSA in the top chamber of the cell-culture inserts, while the bottom chamber was filled with 750 μl of the SFM/1.5% BSA supplemented with 0.1% FCS as chemo-attractant. Cells were then incubated for 22 hours under normal growth conditions. For cell invasion (migration through an ECM coated membrane) cells were prepared as above, but were seeded onto an ECM coated 8 μm pore-sized Matri-Gel invasion chambers (Falcon No. 354480, Le Pont de Claix, France) and cultured for up to 22 hours.

To determine the number of cells migrating (un-coated membrane) or invading (ECM coated membrane) through the membrane, non-migrating or non-invading cells were removed by wiping the top of the membrane with a PBS-wetted cotton-wool tip.

Membranes were then stained using a Hemacolor™ staining kit (Merck, Darmstadt, Germany). Following washing with water, cells migrating or invading through the membrane were manually counted by using magnified (200×) digital pictures of the insert/membranes (4 fields for each membrane). Samples were run in duplicates and four individual fields were counted for each sample. An invasion index was calculated as the number of cells invading through the membrane (cells penetrating the deeper ECM coating) divided by the number of cells migrating through the membrane.

### Example 9

### Statistical Analysis

The Sigma Plot 8.0 graphic program (Jandel Scientific, San Rafael, CA, USA) was used to calculate IGFBP binding affinities for IGF-I and -II, using a one binding-site hyperbolic fit. Each point was measured in quadruplicate. The GraphPad PRISM program was utilised to perform one way ANOVA and Bonferroni post-hoc analysis. All experiments were performed at least three times with samples run in duplicate-quadruplicate, as indicated each time, and results plotted as mean ±SEM

### Example 10

### Initial Results

### Membrane-Bound IGFBP-2 is Displaced by NaCl and EDTA

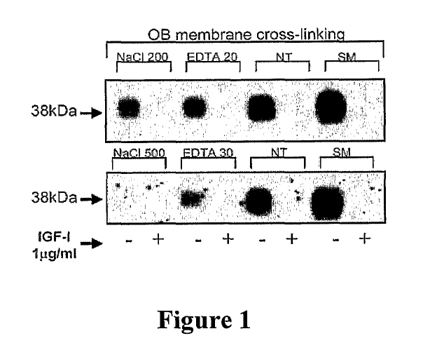

In order to determine whether cell surface association of IGFBP-2 involves ionic interactions, OB membranes were incubated with increasing salt concentration or EDTA prior to cross-linking analysis. NaCl dissociates IGFBP-2 (¹²⁵I-IGF-I cross-linked band at 38 kDa) from the rat brain OB membrane in a dose-dependent manner. The 38 kDa band in FIG. 1 has been previously identified as IGFBP-2 (Russo et al. 1997 (supra)). Dissociation was complete with 500 mM NaCl. EDTA (20-30 mM) also decreased IGFBP-2 binding to OB membranes. These findings suggest that IGFBP-2 binds to OB cell membrane potentially via a Mg⁺⁺/Ca⁺⁺ dependent ionic interaction.

### IGFBP-2 Binds to a Membrane 300 KDa Complex Via GAG Chains

IGFBP-2 binding to membrane PG of >300 kDa, here demonstrated by cross-linking analysis, is decreased (FIG. 2) by chondroitin ABC lyase (Ch-ase) and keratanase (K-ase). The >300 kDa band shown in FIG. 2 is likely to be an ¹²⁵I-IGF-I/IGFBP-2/PG complex. This band of >300 kDa (control lane, FIG. 2) was competed by an excess (1 μg/ml) of cold IGF-I but not by an excess (1 μg/ml) of cold des(1-3)-IGF-I (an IGF-I analogue with reduced affinity for IGFBPs) (Firth et al., 2002 (supra), Russo et al. 1997 (supra)). In these in vitro studies, it is shown that a 38 kDa cross-linked complex, consistent with ¹²⁵I-IGF-I:IGFBP-2, was identified in membranes prepared from rat brain olfactory bulbs. It is also shown that IGFBP-2 binds to cell membrane PGs (>300 kDa) and that IGFBP-2 acts as a linker molecule allowing peri-cellular sequestration of IGF-I in the form of a membrane proteoglycan complex (PG/IGFBP-2/IGF-I). Binding of proteins to the glycosaminoglycan components of proteoglycans is often charge-dependent, although more specific, higher affinity interactions may also occur (Kjellen et al., Annu Rev Biochem 60.443-475, 1991). Since charge-dependent binding to glycosaminoglycans is inhibited by increasing ionic strength, the inhibition of cell-association of IGFBP-2 in a dose-dependent manner by NaCl indirectly supports the hypothesis that IGFBP-2 was binding to glycosaminoglycans. In vitro, IGFBP-2 is known to bind a number of glycosaminoglycans (Firth et al., 2002 (supra), Russo et al. 1997 (supra)) and the proteoglycan aggrecan (Russo et al. 1997 (supra)). Binding of IGFBP-2 to aggrecan is blunted by specific digestion of component glycosaminoglycans (Russo et al. 1997 (supra)). Similarly, PG/IGFBP-2/IGF-I complexes were also blunted by glycosaminoglycan lyases suggesting that IGFBP-2-IGF-I complexes were bound to the glycosaminoglycan component of membrane proteoglycans. It was therefore proposed and tested as set forth in the following experimental sections that the putative heparin-binding domain (HBD) PKKLRP (aa 160-165 in rat; aa 179-184 in human) in the mid-region of IGFBP-2 (Firth et al., 2002 (supra)) are involved.

### Example 11

### Modification of HBD of IGFBP-2 has No Effect on the Affinity of IGFBP-2 for IGFs

In order to investigate the HBD of IGFBP-2, ₁₇₉PKKLRP 84, was mutated to ₁₇₉PNNLAP₁₈₄ and the ₂₆₅RGD₂₆₇ domain was mutated to ₂₆₅RGE₂₆₇. The IGF binding affinities of the mutants were similar to those of native IGFBP-2 (Table 4) indicating that neither substitution of the basic amino acids in the HBD domain, nor ablation of the RGD motif significantly altered the affinity of IGFBP-2 for IGF-I or IGF-II.

### Example 12

### The HBD Mediates Binding of IGFBP-2 to Aggrecan and IGFBP-2 Associates with ECM Components via its HBD

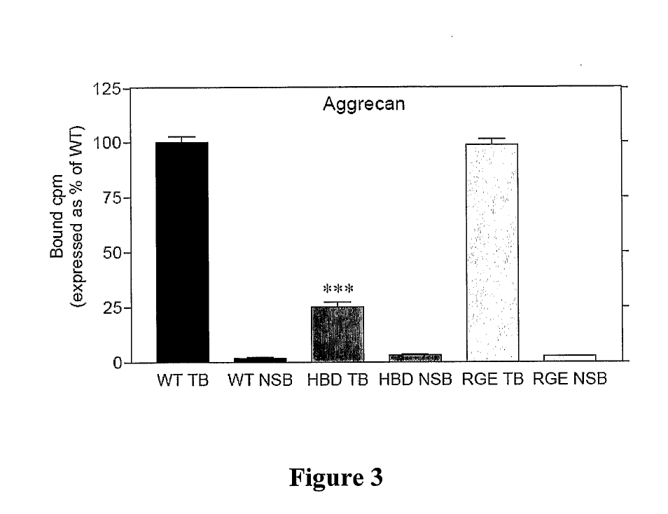

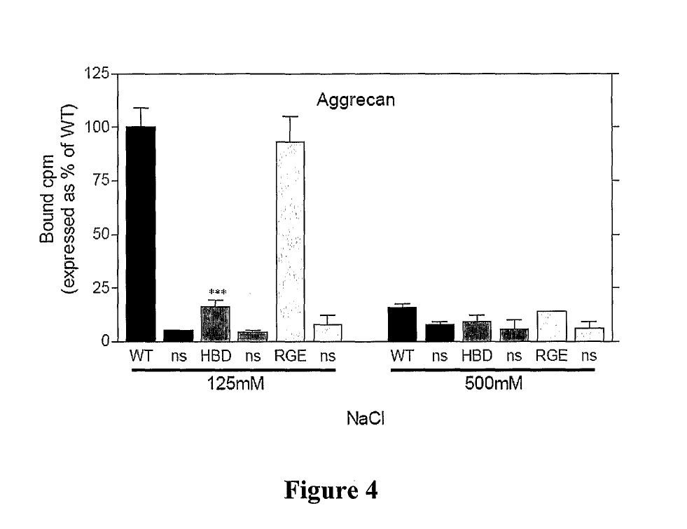

A solid-phase binding assay (Russo et al. 1997 (supra)) was employed to determine whether the introduced mutations would affect IGFBP-2 binding to aggrecan. Preliminary experiments established the time, dose-dependence and optimal amount of coating substrate. Native and RGE-IGFBP-2 mutant, both at 10 ng/well, bound aggrecan to a similar extent, while the HBD-IGFBP-2 mutant, showed a marked 75% reduction in binding under the same conditions (p<0.001, FIG. 3). Binding of native and RGE-IGFBP-2 was decreased by the presence of increasing ionic strength (125-500 mM NaCl, FIG. 4). These results indicate that the 179PKKLRP184 motif mediates most of the IGFBP-2 binding to aggrecan and suggest that ionic interactions are involved. ECM components, including laminin, fibronectin, vitronectin, collagen type IV and proteoglycans, are involved in development of neoplastic processes in the nervous system. Increasing evidence (Russo et al 1997 (supra), Russo et al. Endocrinology 140:3082-3090, 1999, Kricker et al. 2003 (supra), McCaig et al. 2002 (supra)) suggests that IGFBP-2 interacts with components of the extra-cellular matrix (Russo et al. 1997 (supra), Russo et al. 1999 (supra), Kricker et al. 2003 (supra), McCaig et al. 2002 (supra)). It was therefore investigated whether the interaction of IGFBP-2 with matrix components involves the HBD or RGD sequences.

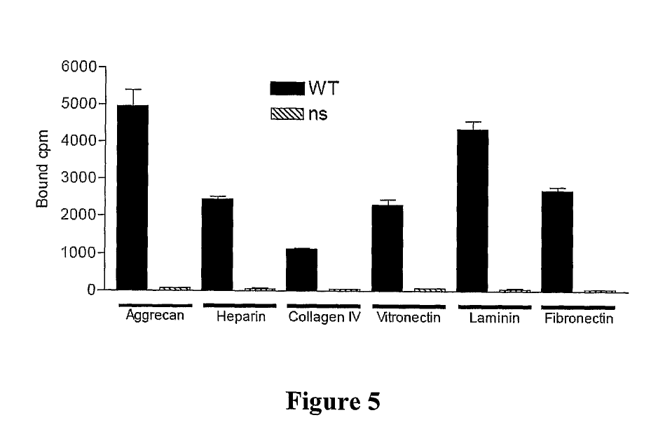

Native IGFBP-2 bound to a variable extent to heparin, vitronectin (VN), laminin (LAM), fibronectin (FN) and collagen type IV (COL-IV) (FIG. 5).

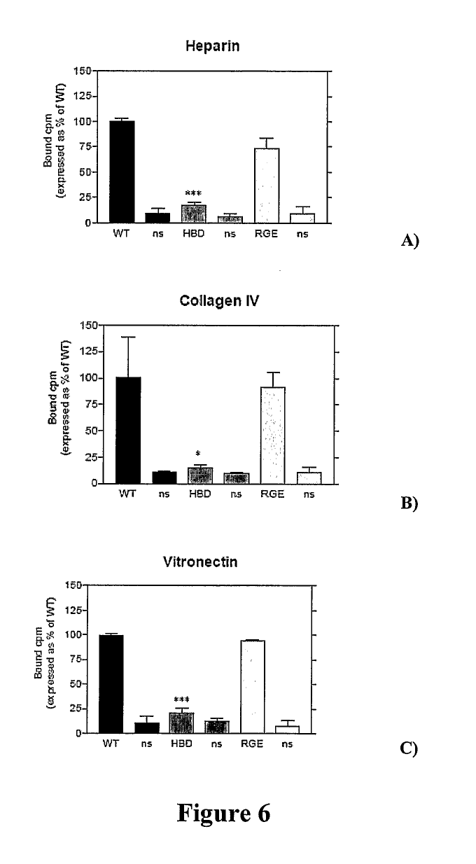

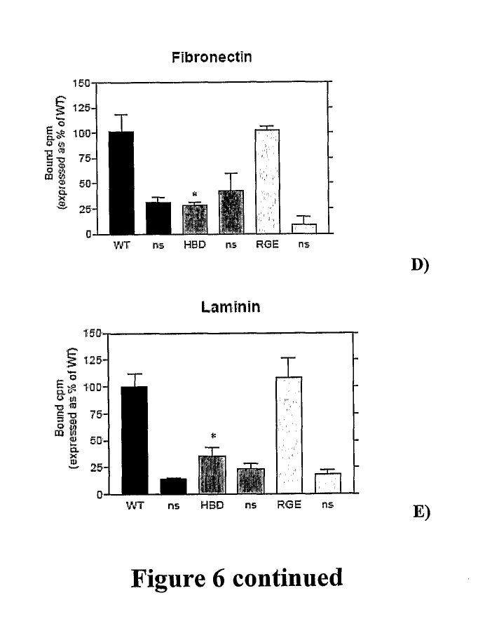

These interactions are mediated by HBD, since the HBD-IGFBP-2 mutant showed markedly reduced (60-80%) binding to all of these substrates (FIG. 6 A-E). However, binding of the RGE-IGFBP-2 mutant to heparin and the ECM components was comparable to that of native IGFBP-2 (FIG. 6A-E). These results show that the HBD but not the RGD motif in IGFBP-2 is involved in interactions with components of the extracellular matrix.

### Example 13

### Exogenous IGFBP-2 and its Modified IGFBP-2 Inhibit the Mitogenic Activity of IGF-I in SHEP Cells

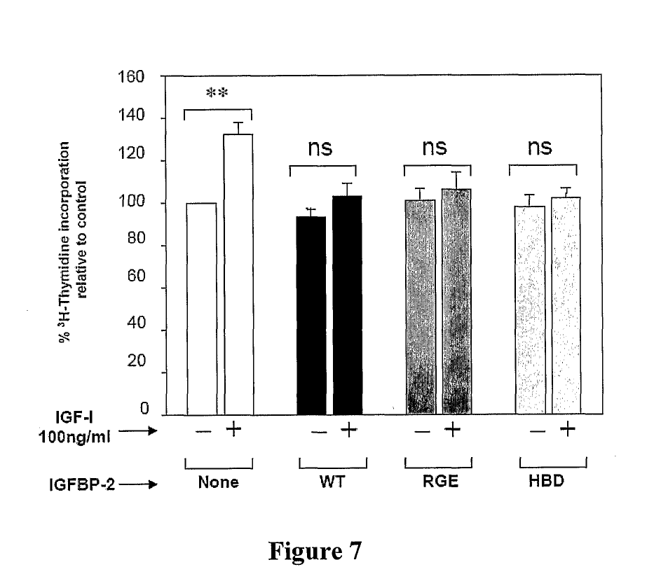

IGFBPs control IGF action at the cellular level by either restricting access to their receptors and thus inhibiting any mitogenic response, or by facilitating IGF-I receptor binding and consequently potentiating the mitogenic stimuli (Firth et al., 2002 (supra)). The effects of IGFBP-2 and its mutants on IGF-1-mediated proliferation were then determined. In SFM, IGF-I alone (100 ng/ml) significantly stimulated proliferation of SHEP cells (p<0.01, FIG. 7). However, this effect was abrogated by the presence of native or mutant IGFBP-2 (each at 800 ng/ml). Addition of either native or mutant IGFBP-2 alone to wild type SHEP cells did not alter basal growth of these cells (FIG. 7).

These data demonstrate that exogenous IGFBP-2 and its mutants, which bind IGF-I with similar affinity, equally inhibit IGF-I actions, and that this inhibition does not require the presence of a functional HBD or RGD domain.

### Example 14

### A Functional HBD is Required for Overexpression of IGFBP-2 to Enhance Neuroblastoma Cells Growth

It has been previously demonstrated that over-expression of IGFBP-2 in vivo negatively regulates postnatal growth, including brain growth, in rodents (Hoeflich et al. 1999 (supra)). On the other hand, IGFBP-2 over-expression may either inhibit or enhance proliferation in various tumor cell lines (Firth et al., 2002 (supra), Moore et al. 2003 (supra), Wang et al. 2003 (supra), Hoeflich et al. Horm Metab Res 35:816-821, 2003). The effects of over-expression of IGFBP-2 or its mutants on SHEP cell proliferation was therefore assessed.

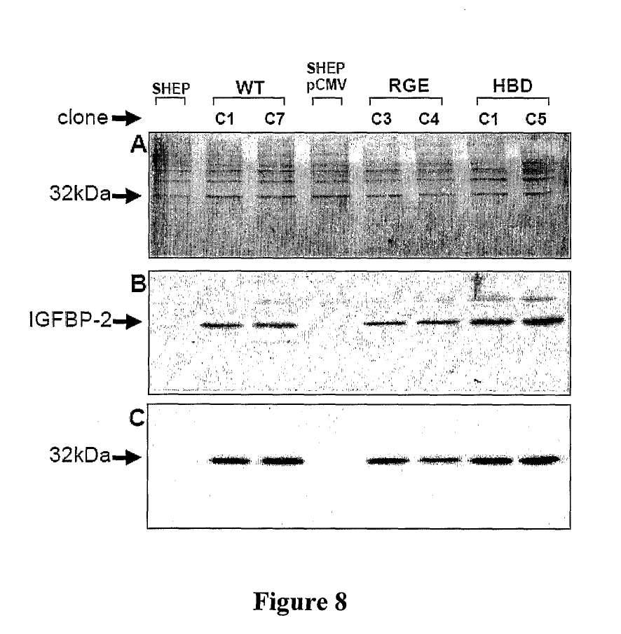

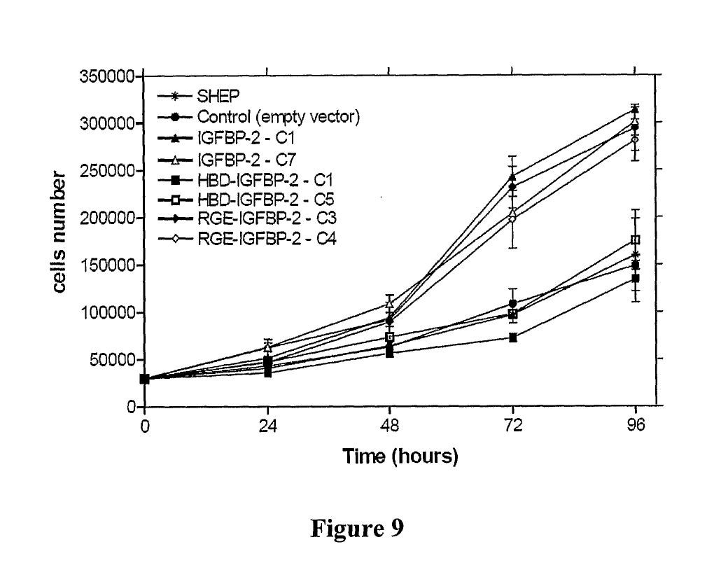

SHEP cell clones expressing comparable amounts of IGFBP-2 (FIG. 8) or its mutants were isolated and grown in complete medium. SHEP cells over-expressing native or RGE-IGFBP-2 showed an 8-fold increase in cell number over the 4 d of culture, compared with a 3-4-fold increase in the SHEP control cells (FIG. 9). Conversely over-expression of HBD-IGFBP-2 did not affect the growth of SHEP cells, which was comparable to that observed in SHEP control cells. Thus an intact HBD is required for overexpression of IGFBP-2 to enhance neuroblastoma cells growth.

### Example 15

### Over-Expression of IGFBP-2 and its HBD Modified form Differentially Modulate IGF-Induced SHEP Cell Proliferation in the Absence of Serum

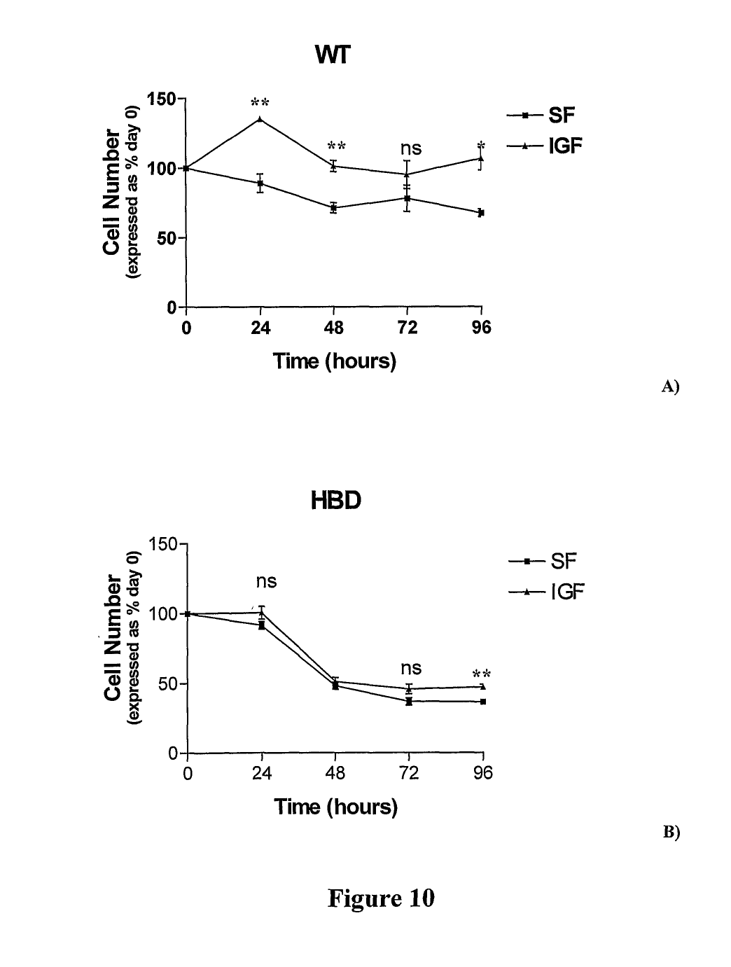

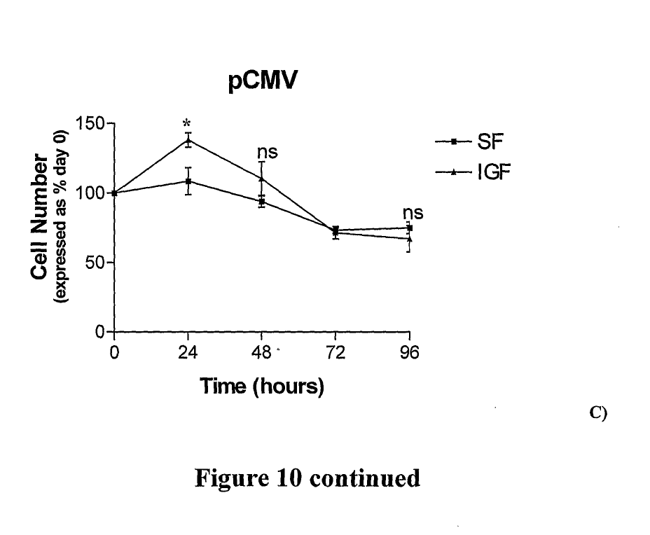

SHEP cell clones expressing comparable amounts of WT-IGFBP-2 or HBD-IGFBP-2 and empty vector control cells (FIG. 8) were cultured in serum free medium in the presence or absence of IGF-I (100 ng/ml, single dose). In serum free medium and in the absence of IGF-I the cell number in the WTIGFBP-2 over-expressing SHEP cells (FIG. 10a, WT) was variably maintained over the 4 days (˜15% decrease). However, under the same conditions, (SF) cell number decreased in the control cells (empty vector, pCMV) by ˜40% (FIG. 10c, pCMV)) and more dramatically (˜60%) in the HBD-IGFBP-2 over-expressing SHEP cells (FIG. 10b, HBD).

Addition of a single dose of IGF elicited proliferation of SHEP cells transfected with either the WT-IGFBP-2 (˜50%, 24 hrs, p<0.001) or empty vector control cells (˜20%, 24 hrs, p<0.05) when compared to the cell number observed in serum free medium and absence of IGF-I at the same time point (FIGS. 10a and c). No response to IGF-I was seen at 24 hrs in HBD-IGFBP-2 over-expressing SHEP cells, with cell number dramatically declining (˜60%), over the 72 hrs, similar to that seen in absence of IGF-I (FIG. 10 b). Since the HBD-IGFBP-2 binds poorly to ECM, these data suggest that IGFBP-2 interactions with ECM are the key to both enhanced cell survival, in serum free media, and potentiation of IGF-I action.

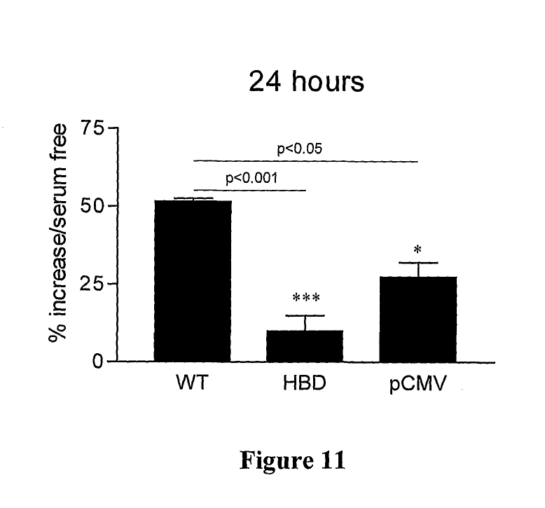

Significant differences in cell number increases over serum free controls were seen among the three SHEP cells clones WT, HBD (p<0.001 versus the WT) and pCMV (p<0.05 versus the WT) at 24 hours as also shown in FIG. 11. Data in FIG. 11 are expressed as percentage increase in cell number induced by IGF-I over serum-free control at time “0”. These data thus show that there is a statistically significant step-wise decrease in the IGF effect from WT to empty vector, to HBD mutant, in which there is no measurable IGF-I effect. The full time course dramatically demonstrates that in the SHEP cells expressing HBD mutant there is no apparent IGF-I effect on cell number, and that the IGF-I effect in control SHEP cells (pCMV) is transient and not maintained

### Example 16

### Exogenously Added HBD-IGFBP-2 Mutant Potently Inhibits Proliferation of Neutoblastoma (SHEP) Cells Over-Expressing Native IGFBP-2

As demonstrated herein IGFBP-2 over-expression enhances proliferation (up to 8 fold) of neuroblastoma SHEP cells, while the growth rate of those over-expressing the heparin binding domain mutant HBD-IGFBP-2 is comparable, if not reduced, to that observed in wild type SHEP control cells (see FIG. 9). Furthermore, over-expression of IGFBP-2 but not its HBD mutant potentiate IGF-induced SHEP cell proliferation in the absence of serum (FIGS. 10 and 11). Over-expression of HBD-IGFBP-2 potently abolished the IGF-I response in SHEP cells. These findings further support the growth inhibitory properties of the HBD-IGFBP-2 mutant. It was then determined whether exposure of the highly proliferating IGFBP-2-SHEP cells, that are a model for malignant cells, to exogenously added purified HBD-IGFBP-2 mutant would inhibit their growth.

WT-IGFBP-2 SHEP cells were grown in DMEM/10% FCS to reach 60% cell confluency, prior to media change with fresh DMEM/10% FCS (Time 0) and further culture for up to 72 hours (Time 72) in the presence or absence (black bars) of 1.5 μg/ml of purified HBD-IGFBP-2 mutant. This quantity of HBD-IGFBP-2 is equivalent to the quantity of native IGFBP-2 expressed/released in the conditioned media by the WT-IGFBP-2 SHEP cells in ˜48 h. This mutant was added at Time 0 or 24 or 48 to allow exposure of WT-IGFBP-2 SHEP cells for the entire 72 hours (grey bars), or just for the last 48 (striped bars) or 24 hours (checked bars). In the latter treatments WT-IGFBP-2 SHEP cells were allowed to proliferate in optimal condition (DMEM/110% FCS) for the first 24 or 48 hours respectively. MTT assays were performed three times and samples were run in triplicate.

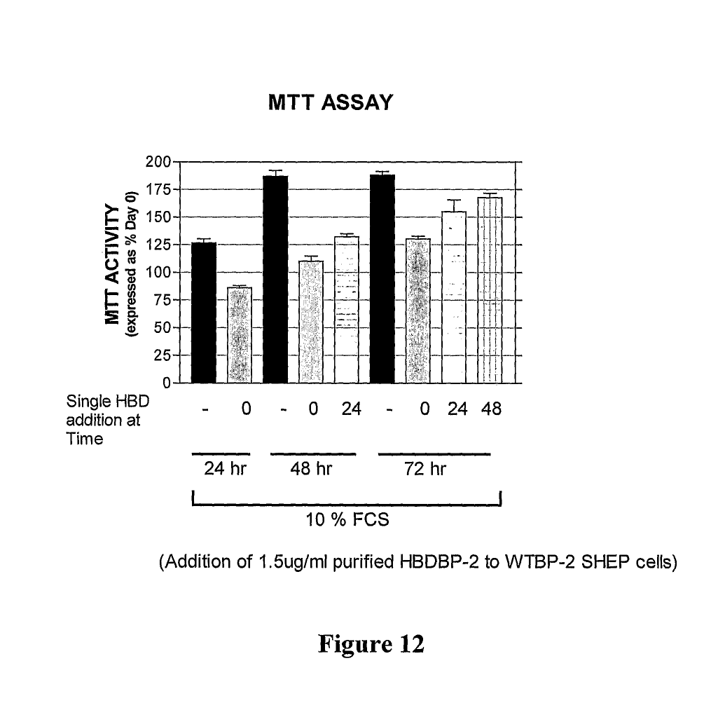

As shown in FIG. 12, proliferation of SHEP cells over-expressing native IGFBP-2 was ablated at 24 hours and strongly inhibited at 48 and 72 hours in the presence of 1.5 μg/ml of HBD-IGFBP-2 (grey bars). Addition of 1.5 μg/ml of HBD-IGFBP-2 to proliferating WT-IGFBP-2-SHEP cells at both Time 24 (striped bars) or 48 (checked bars) also inhibited proliferation of WT-IGFBP-2-SHEP cells. These data show that the HBD-IGFBP-2 mutant is a potent inhibitor of cell proliferation in neuroblastoma cells over-expressing IGFBP-2.

### Example 17

### IGFBP-2 Promotes Migration and Invasion of SHEP Cells Via its HBD

It was then determined whether addition or over-expression of IGFBP-2 and its modified forms affects metastatic parameters including migration and invasion.

Addition of native or RGE-IGFBP-2 (1.6 μg/ml) to wild type SHEP cells significantly (˜25%, p<0.05) enhanced their migration/motility through the uncoated membrane (data not shown). In contrast, addition of the HBD-IGFBP-2 mutant did not affect migration/motility of wild type SHEP cells (data not shown).

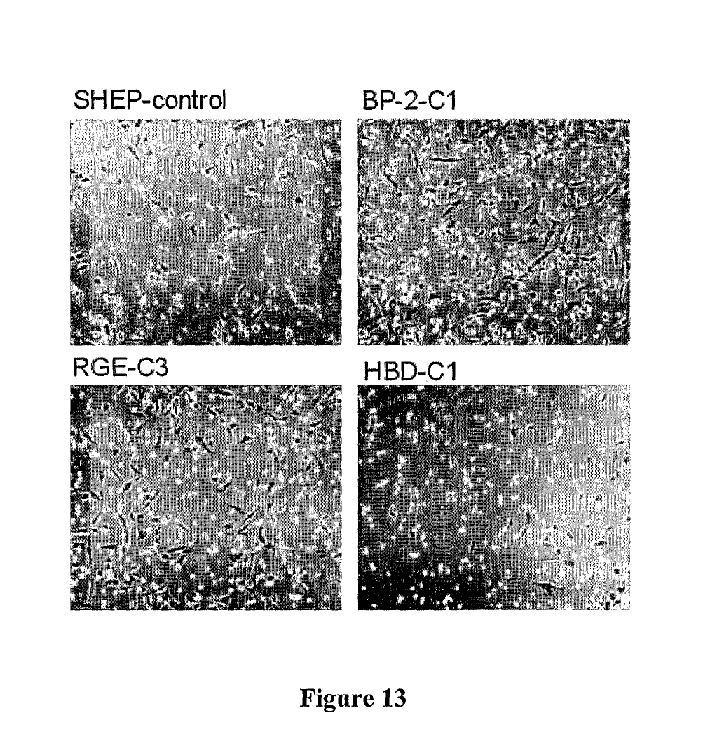

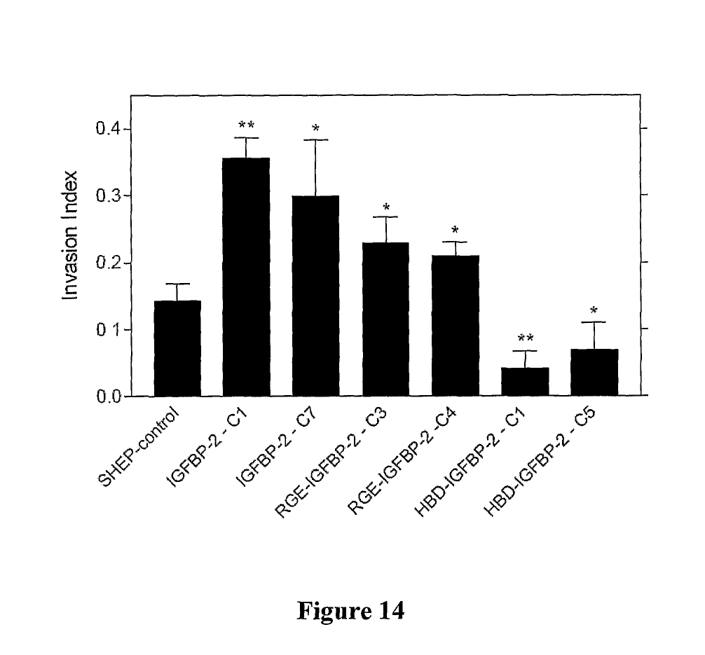

Over-expression of either WT or RGE IGFBP-2 dramatically increased SHEP cell invasion throughout ECM coated membranes by 2 3-fold over that of the SHEP cell controls (native p<0.01, RGE p<0.05, FIGS. 13 and 14). In contrast, invasion of SHEP cell clones expressing the HBD-IGFBP-2 mutant was significantly decreased by 50-70% compared to that of the control-SHEP cells (FIGS. 13 and 14). Thus, an intact HBD is required for IGFBP-2 to enhance neuroblastoma cell migration, whether IGFBP-2 is added exogenously or over-expressed.

### Example 18

### Summary

The present studies specifically mutated the HBD and RGD motifs of IGFBP-2 to determine their roles in IGFBP-2 binding to ECM, and proliferation, migration and invasion of neuroblastoma cells. It is demonstrated herein for the first time that mutation of the HBD but not the RGD motif inhibits binding of IGFBP-2 to ECM components. These results are consistent with those previously reported for IGFBP-3 and/or IGFBP-5 binding to ECM (Booth et al. Growth Regulation 5:1-17, 1995, Campbell et al., Am J Physiol 273:E1005-13, 1997, Gui et al., J. Clin. Endocrinol. Metab., 86:2104-2110, 2001, Nam et al. Endocrinology 138.2972-2978, 1997, Nam et al. Endocrinology 141:1100-1106, 2000, Nam et al. Endocrinology 143:30-36, 2002, Arai et al. Journal of Biological Chemistry 271:6099-6106, 1996, Firth et al. Journal of Biological Chemistry 273.2631-2638, 1998). However the HBDs of these IGFBPs, involved in these interactions, are located in their C-terminal domains, while the HBD in IGFBP-2, which is mutated in this studies, is located in the linker region (Shimasaki et al., Progress in Growth Factor Research 3:243-266, 1991).

Addition of IGFBP-2 resulted in inhibition of IGF-I-stimulated neuroblastoma cell growth. This effect was independent of HBD or RGD motifs. However, over-expression of IGFBP-2 or RGE-IGFBP-2 in neuroblastoma cells resulted in dramatically enhanced cell proliferation, whereas over-expression of the HBD-IGFBP-2 mutant, with reduced binding to extracellular matrix components, did not result in any growth advantage. The critical difference between the contrasting effects on proliferation of exogenously added and over-expressed IGFBP-2 is thus the presence of an intact HBD domain. This suggests that the growth inhibition of exogenous IGFBP-2 is entirely explained by its sequestration of IGF-I since the mutants have similar IGF-I binding affinity to native IGFBP-2. In contrast, the growth enhancement of over-expressed IGFBP-2 only occurs in the presence of an intact HBD domain, suggesting that it is dependent on its association with peri-cellular matrix proteins and/or proteoglycans or glycosaminoglycans at the cell surface.

In contrast to the effects on proliferation, addition or over-expression of native or RGE-IGFBP-2 significantly enhanced invasion of neuroblastoma cells, while HBD-IGFBP-2 over-expression strongly inhibited SHEP neuroblastoma cell invasion. These results shows that a functional HBD in IGFBP-2 is required for modulation of this process.

The migratory behaviour of cells is fundamental to tumor metastasis. One of the major features of metastatic cells is the re-organisation of specific membrane components (e.g. integrin receptors, proteoglyeans) and activation of specific enzymatic processes (e.g. matrix proteases) which allow cell migration and invasion through the extracellular matrix (Stewart et al. Reprod Biol Endocrinol 2.2, 2004, Zhang et al. Horm Metab Res 35:802-808, 2003, Nagle J Cell Biochem 91:36-40, 2004, Hojilla et al. Br J Cancer 89:1817-1821, 2003). IGFBP-2 appears to be involved in metastatic processes via both IGF-dependent and independent mechanisms in meningiomas (Nordqvist et al., J Neurooncol 57:19-26, 2002), prostate cancer (Moore et al. 2003 (supra), Shariat et al. J Clin Oncol 20:833-841, 2002), ovarian cancer (Baron-Hay et al. Clin Cancer Res 10.1796-1806, 2004), melanocytic lesions (Wang et al. 2003 (supra)) and gliomas (Song et al. Proc Natl Acad Sci USA 100:13970-13975, 2003, Wang et al. 2003 (supra)). IGFBP-2 contributes to glioma progression in part by enhancing matrix metalloprotease-2 (MMP-2) gene transcription and tumor cell invasion (Wang et al. 2003 (supra)).

IGFs are likely to play a minimal if any role in the effect of IGFBP-2 on migration, which appears to be dependent on its HBD and therefore the ability of IGFBP-2 to adhere to the pericellular matrix. While the proliferation-enhancing effect of IGFBP-2 is also dependent on its ability to bind to the pericellular matrix, this process appears far more likely to involve IGF targeting to its receptors, since exogenous IGFBP-2, via IGF sequestration, negates the effect. It is proposed that the HBD is involved in a number of key biological functions of IGFBP: i) interaction with components of the peri- and extra-cellular matrix; ii) peri-cellular sequestration of local IGF-I in control of cellular growth; iii) IGF-independent activation of invasive or metastatic processes. It is shown herein, in neuroblastoma cells that IGFBP interacts with components of the extra-cellular matrix via its heparin-binding-domain. Furthermore, IGFBP enhances neuroblastoma cell migration and invasion, a function that directly or indirectly utilizes the heparin-binding domain. Since the RGE mutant behaved in a similar fashion to native IGFBP in all assays performed, the RGD (wild type) sequence is not involved in binding to ECM components, proliferation or invasion. These findings thus point to a key functions for the HBD of IGFBP in the control and regulation of a number of developmental and disease process of the nervous system including neuroblastoma growth and migration. These studies therefore significantly contribute to understanding the mechanisms whereby IGFBP may enhance tumor growth and metastasis. This information will provide the potential for rational therapeutic manipulation of the pro-carcinogenic activities of IGFBP in neuroblastoma and other related malignancies.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 4

| | IGF-I | IGF-II |
|---|---|---|
| | H_{D}[nM] | H_{D}[nM] |
| IGFBP-2 | 1.14 ± 0.31 | 0.37 ± 0.11 |
| HBD-IGFBP-2 | 1.45 ± 0.35 | 0.52 ± 0.16 |
| RGE-IGFBP-2 | 1.13 ± 0.26 | 0.66 ± 0.11 |

### BIBLIOGRAPHY

Arai et al. Journal of Biological Chemistry 271.6099-6106, 1996.
Arkin et al., Proc Natl Acad Sci USA 89:7811-7815, 1992.
Atherton et al., Peptide Synthesis, Chapter 9, “Synthetic Vaccines” edited by Nicholson and published by Blackwell Scientific Publications.
Ausubel et al. Current Protocols in Molecular Biology, 5^{th} Edition, John Wiley & Sons, Inc, N.Y., 2002.
Bach et al., Diabetes Review 3:38-61, 1995.
Bacher et al., Drug Discovery Today, 3(6):265-273, 1998.
Bandyopadhyay et al., Mol. Cell. Biol., 4:749-754, 1984.
Baron-Hay et al. Clin Cancer Res 10:1796-1806, 2004.
Berglund et al., Biotechnology, 11.916-920, 1993.
Berkner et al., BioTechniques, 6:616-629, 1988.
Berkner, Curr. Top. Microbiol. Immunol., 158.39-66, 1992.
Bernfield et al. Annu Rev Biochem 68:729-777, 1999.
Binoux et al. Contemporary Endocrinology. The IGF system pp 281-313, 1999.
Booth et al. Growth Regulation 5:1-17, 1995.
Breakefield et al., Mol. Neurobiol., 1.339-371, 1987.
Buchschacher et al., J. Virol., 66.2731-2739, 1982.
Burton et al., Proc. Natl. Acad. Sci. USA., 88:10134, 1991.
Campbell et al., Am J Physiol 273:E1005-13, 1997.
Chaudhary et al. Proc Natl Acad Sci USA. 87:1066-1070, 1990.
Clackson et al. Nature 352:624, 1991.
Coligan et al. Current Protocols In Protein Science (John Wiley & Sons, Inc. 1995-1997.
Culver, Gene Therapy: A Primer for Physicians, 2^{nd} Ed., Mary Ann Liebert, 1996.
Dayhoff et al. Atlas of Protein Sequence and Structure (Natl Biomed Res Found, Washington, D.C., 1978.
Delgrave et al. Protein Engineering 6:327-331, 1993.
Elghanian et al., Science, 277:1078-1081, 1997.
Ellington et al., Nature 346:818, 1990.
Elmlinger et al. Endocrinology 142.1652-1658, 2001.
Elmlinger et al. Growth Regul 6:152-157, 1996.
Erickson et al. Science 249:527-533, 1990.
Fink et al., Hum. Gene Ther., 3:11-19, 1992.
Fink et al., Ann. Rev. Neurosci., 19.265-287, 1996.
Firth et al., Endocr Rev 23:824-854, 2002.
Firth et al. Journal of Biological Chemistry 273.2631-2638, 1998.
Fodor et al. Science 251:767, 1991.
Friedman, In: Therapy for Genetic Disease, T. Friedman, Ed., Oxford University Press, pp. 105-121, 1991.
Freese et al., Biochem. Pharmacol., 40.2189-2199, 1990.
Gold et al., Annu. Rev. Biochem., 64:763-797.1995.
Gonnet et al. Science 256(5062):1443-1445, 1992.
Gorziglia et al., J. Virol., 66:4407-4412, 1992.
Grompe et al., Proc. Natl. Acad. Sci. USA, 86.5855-5892, 1989.
Grompe, Proc. Natl. Acad. Sci. USA, 86:5855-5892, 1993.
Gui et al., J Clin Endocrinol Metab 86.2104-2110, 2001.
Harlow et al., Antibodies: A Laboratory Manual, Cold Spring Harbor Laboratory, 1988.
Helseth et al., J. Virol., 64.2416-2420, 1990.
Ho et al., Endocrinology 138:3811-3818, 1997.
Hodgson Bio/Technology 9.19-21, 1991.
Hoeflich et al. Biochem Biophys Res Commun 324:705-10, 2004.
Hoeflich et al. Endocrinology 140.5488-5496, 1999.
Hoeflich et al. Horm Metab Res 35:816-821, 2003.
Hojilla et al. Br J Cancer 89.1817-1821, 2003.
Hoogenboom et al. Nucleic Acids Res., 19:4133, 1991.
Hossenlopp et al. Analytical Biochemistry 154.138-143, 1986.
Huston et al. Proc Natl Acad Sci U.S.A. 85:5879-5883, 1988.
Iozzo, Ann Rev Biochem 67:609-652, 1998.
Jayasena, Clin. Chem., 45(9):1628-1650, 1999.
Johnson et al. “Peptide Turn Mimetics” in Biotechnology and Pharmacy.
Johnson et al., J. Virol., 66.2952-2965, 1992.
Jones et al., Endocrine Reviews 16:3-34, 1995.
Kang et al., Proc. Natl. Acad. Sci. U.S.A., 88:4363, 1991.
Kjellen et al., Annu Rev Biochem 60.443-475, 1991.
Kricker et al. Endocrinology 144:2807-2815, 2003.
Kunkel et al. Methods in Enzymol 154:367-382, 1987.
Kunkel, Proc Natl Acad Sci USA 82:488-492, 1985.
Lowman et al. Biochemistry 30:10832, 1991.
McCaig et al. J Cell Sci 115.4293-4303, 2002.
Madzak et al., J. Gen. Virol., 73:1533-1536, 1992.
Mann et al., J. Virol., 54:401-407, 1985.
Margolskee, Curr. Top., Microbiol. Immunol., 158.67-95, 1992.
Miller et al., Mol. Cell. Biol., 5:431-437, 1985.
Miller et al., J. Virol., 62:4337-4345, 1988.
Miller, Curr. Top. Microbiol. Immunol., 158:1-24, 1992.
Modrich, Ann. Rev. Genet., 25:229-253, 1991.
Moore et al. Int J Cancer 105:1419-2003.
Morris et al., Proc. Natl. Acad. Sci., USA, 95(6):2902-2907, 1998.
Mosmann, J. Immunol. Methods, 16:65(1-2):55-63, 1983.
Moss, Curr. Top. Microbiol. Immunol., 158.25-38, 1992.
Moss, Proc. Natl. Acad. Sci. USA, 93:11341-11348, 1996.
Muzyczka, Curr. Top. Microbiol. Immunol., 158.97-129, 1992.
Nagle J Cell Biochem 91:36-40, 2004.
Naldini et al., Science, 272:263-267, 1996.
Nam et al. Endocrinology 138:2972-2978, 1997.
Nam et al. Endocrinology 141:1100-1106, 2000.
Nam et al. Endocrinology 143:30-36, 2002.
Nordqvist et al. Cancer Research 57:2611-2614, 1997.
Nordqvist et al., J Neurooncol 57: 19-26, 2002.
O'Connell et al., Am. J. Hum. Genet., 63:259-266, 1998.
Oh et al. Journal of Biological Chemistry 268:14964-14971, 1993.
Oh et al. Journal of Biological Chemistry 268:26045-26048, 1993.
Ohi et al., Gene, 89:279-282, 1990.
Orita et al., Proc. Nat. Acad. Sci. USA, 86.2776-2770, 1989.
Page et al., J. Virol., 64:5270-5276, 1990.
Parker et al. Journal of Biological Chemistry 271: 13523-13529, 1996.
Parker et al. Molecular Biology of the Cell 9:2383-2392, 1998.
Pereira et al. Cancer Res 64:977-984, 2004.
Petropoulos et al., J. Viol., 66:3391-3397, 1992.
Pezzuto et al. Eds., Chapman and Hall, New York, 1993.
Quantin et al., Proc. Natl. Acad. Sci. USA, 89:2581-2584, 1992.
Radulescu, Trends in Biochemical Science 19:278, 1994.
Rajaram et al. Endocrine Reviews 18:801-831, 1997.
Rees et al., Journal of Cellular Biochemistry 71.375-381, 1998.
Remington's Pharmaceutical Sciences, 18^{th} Ed. (1990, Mack Publishing, Company, Easton, Pa., U.S.A.
Roberge et al. Science 269:202, 1995.
Rosenfeld et al., Cell, 68.143-155, 1992.
Russell et al., Nature Genetics, 18:323-328, 1998.
Russo et al. Endocrinology 138:4858-4867, 1997.
Russo et al. Endocrinology 140:3082-3090, 1999.
Sallinen et al. Cancer Res 60:6617-6622, 2000.
Sambrook, Molecular Cloning: A Laboratory Manual, 3^{rd} Edition, CSHLP, CSH, NY, 2001.
Sambrook, Chapter 13, Molecular Cloning, 2001.
Schedlich et al. Journal of Biological Chemistry 273: 18347-18352, 1998.
Schneider et al., Nature Genetics, 18:180-183, 1998.
Schutt et al. J Mol Endocrinol 32:859-68, 2004.
Shariat et al. J Clin Oncol 20:833-841, 2002.
Sheffield et al., Am. J. Hum. Genet., 49:699-706, 1991.
Shimada et al., J. Clin. Invest., 88:1043-1047, 1991.
Shimasaki et al., Progress in Growth Factor Research 3:243-266, 1991.
Song et al. Proc Natl Acad Sci USA 100:13970-13975, 2003.
Sorge et al., Mol. Cell. Biol., 4:1730-1737, 1984.
Stewart et al. Reprod Biol Endocrinol 2:2, 2004.
Stratford-Perricaudet et al., Hum. Gene Ther., 1:241-256, 1990.
Summerton et al., Antisense and Nucleic Acid Drug Development 7:187-195, 1997.
Tuerk et al., Gold Science 249:505, 1990.
Wang et al. Cancer Res 63:4315-4321, 2003.
Wang et al. J Cutan Pathol 30:599-605, 2003.
Watson et al. (Molecular Biology of the Gene, Fourth Edition, Benjamin/Cummings, Menlo Park, Calif., 1987.
Wells, Methods Enzymol 202.2699-2705, 1991.
White et al., Genomics, 12:301-306, 1992.
Wilkinson et al., Nucleic Acids Res., 20:2233-2239, 1992.
Winter et al. Annual Review of Immunology 12:433-455, 1994.
Wraight et al. Journal of Investigative Dermatology 111:239-242, 1998.
Yamanaka et al. Endocrinology 140:1319-1328, 1999.
Zhang et al. Brain Pathol 12.87-94, 2002.
Zhang et al. Horm Metab Res 35:802-808, 2003.
Zubay, Biochemistry third edition, Wm. C. Brown Publishers, 1993.

1. A method for modulating cellular proliferation, invasion and/or migration comprising regulating the interaction between a Heparin Binding Domain (HBD) of Insulin-Like Growth Factor Binding Protein (IGFBP) and the extracellular (EC) or pericellular (PC) matrix.

2. The method of claim 1 comprising administering an effective amount of an agent to a cell to down regulate the interaction between IGFBP and the EC or PC matrix and reduces cell proliferation, invasion and/or migration.

3. The method of claim 2 wherein the agent is small or large chemical molecule, polypeptide or nucleic acid molecule.

4. The method of claim 2 wherein the agent is a modified IGFBP polypeptide comprising a modified HBD which exhibits reduced binding to EC or PC matrix or wherein the agent is a genetic sequence from which the modified polypeptide is producible.

5. The method of claim 3 wherein the modified IGFBP is further modified and exhibits a reduced or no ability to bind Insulin-like Growth Factor.

6. The method of claim 2 wherein the agent binds to and down regulates the activity of a HBD of IGFBP.

7. The method of claim 6 wherein the agent is a peptide comprising the amino acid sequence of an HBD of IGFBP-2 or comprising a functional variant of the amino acid sequence of an HBD of IGFBP-2.

8. The method of claim 6 wherein the agent is an antibody or aptamer.

9. The method of claim 2 wherein the agent binds to and down regulates the activity of an Insulin-Like Growth Factor Binding Protein Binding Domain (IBD) of the EC or PC matrix.

10. The method of claim 9 wherein the agent is a peptide comprising the amino acid sequence of an IBD of a matrix component selected from the group consisting of aggrecan, laminin, fibronectin, vitronectin, collagen type IV, proteoglycan, glycosaminoglycan, and mucopolysaccharide structures including heparin, or wherein the agent comprises a functional variant of an IBD of a matrix component selected from the group consisting of aggrecan, laminin, fibronectin, vitronectin, collagen type IV, proteoglycan, glycosaminoglycan, and mucopolysaccharide structures including heparin.

11. The method of claim 2 wherein the cell is a neural cell.

12. The method of claim 2 wherein the cell is an olfactory bulb cell.

13. The method of claim 2 wherein the cell is a tumor cell.

14. The method of claim 10 wherein the tumor cell is a cancerous cell.

15. The method of claim 13 wherein the tumor is a neuroepithelial tumor.

16. The method of claim 1, wherein the EC or PC matrix component is selected from the group consisting of aggrecan, laminin, fibronectin, vitronectin, collagen type IV, proteoglycan, glycosaminoglycan and mucopolysaccharide structures including heparin.

17. A method for treating a tumor cell in a subject comprising administering to the subject an effective amount of an agent to down regulate the interaction between a HBD of IGFBP-2 and the EC or PC matrix and thereby reduce cellular proliferation, invasion and/or migration of the tumor cell.

18. The method of claim 17 wherein the agent is small or large chemical molecule, polypeptide or nucleic acid molecule.

19. The method of claim 17 wherein the agent is a modified IGFBP-2 polypeptide comprising a modified HBD which exhibits reduced binding to EC or PC matrix or wherein the agent is a genetic sequence from which the modified polypeptide is producible.

20. The method of claim 18 wherein the modified IGFBP-2 is further modified and exhibits a reduced or no ability to bind Insulin-like Growth Factor.

21. The method of claim 17 wherein the agent binds to and down regulates the activity of a HBD of IGFBP-2.

22. The method of claim 21 wherein the agent is a peptide comprising the amino acid sequence of an HBD of IGFBP-2 or comprising a functional variant of the amino acid sequence of an HBD of IGFBP-2.

23. The method of claim 21 wherein the agent is an antibody or aptamer.

24. The method of claim 17 wherein the agent binds to and down regulates the activity of an Insulin-Like Growth Factor Binding Protein Binding Domain (IBD) of the EC or PC matrix.

25. The method of claim 24 wherein the agent is a peptide comprising the amino acid sequence of an IBD of a matrix component selected from the group consisting of aggrecan, laminin, fibronectin, vitronectin, collagen type IV, proteoglycan, glycosaminoglycan, and mucopolysaccharide structures including heparin, or wherein the agent is a peptide comprising a functional variant of an IBD of a matrix component selected from the group consisting of aggrecan, laminin, fibronectin, vitronectin, collagen type IV, proteoglycan, glycosaminoglycan, and mucopolysaccharide structures including heparin.

26. The method of claim 17 wherein the cell is a neural cell.

27. The method of claim 17 wherein the cell is an olfactory bulb cell.

28. The method of claim 26 wherein the cell is a tumor cell.

29. The method of claim 25 wherein the tumor cell is a cancerous cell.

30. The method of claim 28 wherein the tumor is a neuroepithelial tumor.

31. The method of claim 17 wherein the EC or PC matrix component is selected from the group consisting of aggrecan, laminin, fibronectin, vitronectin, collagen type IV, proteoglycan, glycosaminoglycan, and mucopolysaccharide structures including heparin.

32. The method of claim 17 wherein the agent reduces or prevents tumor cell metastases.

33. (canceled)

34. (canceled)

35. (canceled)

36. The method of claim 13 wherein said tumor is a neuroepithelial tumor selected from the group consisting of an astrocytoma, gliomas, oligodenroglioma, spongioblastoma, ependymoma, medulloblastoma, neuroblastoma, choroid plexus papilloma, ganglioma, gangliocytomas and pineal tumors, or wherein said tumor is a non-neuroepithelial tumor selected from the group consisting of a meningioma, pituitary tumor, craniopharyngioma, neurilemmoma, schwannoma, acoustic neuroma, melanoma, CNS lymphoma, chordomas Rathke Cleft Cyst, brain metastasis and a leptomeningeal carcinomatose.

37. The method of claim 28, wherein said tumor is a neuroepithelial tumor selected from the group consisting of an astrocytoma, gliomas, oligodenroglioma, spongioblastoma, ependymoma, medulloblastoma, neuroblastoma, choroid plexus papilloma, ganglioma, gangliocytomas and pineal tumor, or wherein said tumor is a non-neuroepithelial tumor selected from the group consisting of a meningioma, pituitary tumor, craniopharyngioma, neurilemmoma, schwannoma, acoustic neuroma, melanoma, CNS lymphoma, chordomas Rathke Cleft Cyst, brain metastasis, and a leptomeningeal carcinomatose.


SEQUENCE LISTING (13 sequences)

SEQ ID NO: 1 - DNA, 1439 bp, Homo sapiens
tgcggcggcgagggaggaggaagaagcggaggaggcggctcccgcgctcgcagggccgtgccacctgcccgcccgcccgctcgctcgctcgcccgccgcgccgcgctgccgaccgccagcatgctgccgagagtgggctgccccgcgctgccgctgccgccgccgccgctgctgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcccggctggagggcgaggcgtgcggcgtctacaccccgcgctgcggccaggggctgcgctgctatccccacccgggctccgagctgcccctgcaggcgctggtcatgggcgagggcacttgtgagaagcgccgggacgccgagtatggcgccagcccggagcaggttgcagacaatggcgatgaccactcagaaggaggcctggtggagaaccacgtggacagcaccatgaacatgttgggcgggggaggcagtgctggccggaagcccctcaagtcgggtatgaaggagctggccgtgttccgggagaaggtcactgagcagcaccggcagatgggcaagggtggcaagcatcaccttggcctggaggagcccaagaagctgcgaccaccccctgccaggactccctgccaacaggaactggaccaggtcctggagcggatctccaccatgcgccttccggatgagcggggccctctggagcacctctactccctgcacatccccaactgtgacaagcatggcctgtacaacctcaaacagtgcaagatgtctctgaacgggcagcgtggggagtgctggtgtgtgaaccccaacaccgggaagctgatccagggagcccccaccatccggggggaccccgagtgtcatctcttctacaatgagcagcaggaggctcgcggggtgcacacccagcggatgcagtagaccgcagccagccggtgcctggcgcccctgccccccgcccctctccaaacaccggcagaaaacggagagtgcttgggtggtgggtgctggaggattttccagttctgacacacgtatttatatttggaaagagaccagcaccgagctcggcacctccccggcctctctcttcccagctgcagatgccacacctgctccttcttgctttccccgggggaggaagggggttgtggtcggggagctggggtacaggtttggggagggggaagagaaatttttatttttgaacccctgtgtcccttttgcataagattaaaggaaggaaaagtaaa

SEQ ID NO: 2 - protein, 328 aa, Homo sapiens
MLPRVGCPALPLPPPPLLPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 3 - DNA, 22 bp, Artificial Sequence
  oligonucleotide
gaaggaggcctggtggagaacc

SEQ ID NO: 4 - DNA, 21 bp, Artificial Sequence
  oligonucleotide
ccgggaagctgatccagggag

SEQ ID NO: 5 - DNA, 40 bp, Artificial Sequence
  oligonucleotide
gcctggaggagcccaacaacctggcaccaccccctgccag

SEQ ID NO: 6 - DNA, 40 bp, Artificial Sequence
  oligonucleotide
ctggcagggggtggtgccaggttgttgggctcctccaggc

SEQ ID NO: 7 - DNA, 23 bp, Artificial Sequence
  oligonucleotide
accatccggggggaacccgagtg

SEQ ID NO: 8 - DNA, 29 bp, Artificial Sequence
  oligonucleotide
caaccggtctactgcatccgctgggtgtg

SEQ ID NO: 9 - DNA, 27 bp, Artificial Sequence
  oligonucleotide
ctcgagatgctgccgagagtcggctgc

SEQ ID NO: 10 - DNA, 42 bp, Artificial Sequence
  oligonucleotide
ctgagcagcacgcggcgatgggcgggggtgcgcatcaccttg

SEQ ID NO: 11 - DNA, 42 bp, Artificial Sequence
  oligonucleotide
caaggtgatgcgcacccccgcccatcgccgcgtgctgctcag

SEQ ID NO: 12 - protein, 6 aa, Artificial Sequence
  heparin binding domain
PKKLRP

SEQ ID NO: 13 - protein, 6 aa, Artificial Sequence
  modified heparin binding domain
PNNLAP